(19) United States
(12) Patent Application Publication
(10) Pub. No.: US 2025/0182846 A1
(43) Pub. Date: Jun. 5, 2025

(54) Identifying microbial gene expression in human tissues

(71) Applicant: The Wistar Institute of Anatomy and Biology, Philadelphia, PA (US)

(72) Inventor: Noam Auslander, Philadelphia, PA (US)

(21) Appl. No.: 18/970,052

(22) Filed: Dec. 5, 2024

Related U.S. Application Data
(60) Provisional application No. 63/606,553, filed on Dec. 5, 2023.

(51) Int. Cl. G16B 20/00; G06F 30/27; G16B 25/10; G16B 40/20

(52) U.S. Cl. CPC G16B 20/00; G06F 30/27; G16B 25/10; G16B 40/20

(57) ABSTRACT

The present invention relates to a tool to efficiently detect viral and bacterial expression in human tissues through RNAseq. The invention employs a neural network to predict reads of likely microbial origin, which are targeted for assembly into longer contigs, improving identification of microbial species and genes. In some embodiments, the invention is applied to perform a systematic comparison of bacterial expression in ESCA and healthy esophagi.

### CROSS-REFERENCE TO RELATED APPLICATIONS

This application claims priority to U.S. Provisional Patent Application No. 63/606,553, filed Dec. 5, 2023, the contents of which are incorporated by reference herein in its entirety.

### STATEMENT REGARDING FEDERALLY SPONSORED RESEARCH OR DEVELOPMENT

This invention was made with government support under CA252025 awarded by National Institutes of Health. The government has certain rights in the invention.

### BACKGROUND OF THE INVENTION

Esophageal carcinoma (ESCA) is among the most common cancers, with around 600,000 new cases diagnosed each year (Yang et al., Front Oncol. (2020) 10:1727; Li et al., Chin J Cancer Res. (2021) 33:535-47). The five-year survival rate for esophageal cancer patients is low, with estimates ranging across populations from 15% to 24%, and is markedly lower than the survival rates of patients with other common gastrointestinal cancers, such as stomach (21-33%) and colon (59-71%) cancers (Arnold et al., Lancet Oncol. (2019) 20:1493-505). While some lifestyle factors, such as smoking, are known to contribute to the development of ESCA, the causes and risk factors remain incompletely characterized (Li et al., Chin J Cancer Res. (2021) 33:535-47). Like other organs of the gastrointestinal tract, the healthy esophagus has a substantial resident bacterial population, principally members of Streptococcus and a handful of other genera (Corning et al., Curr Gastroenterol Rep. (2018) 20:39; Park et al., J Neurogastroenterol Motil. (2020) 26:171-9). Yet, shifts in the esophageal microbiome have been associated with the development of esophageal cancer and of a precursor condition called Barrett's esophagus (Lv et al., World J Gastroentrol. (2019) 25:2149-61). Beyond microbiome shifts, several bacterial species in the colon are thought to be oncogenic in colorectal cancer, such as Streptococcus bovis, Bacteroides fragilis, and Fusobacterium nucleatum (Cheng et al., Front Immunol. (2020) 11:615056; Pignatelli et al., Microorganisms. (2023) 11:2358). F. nucleatum is also a pathogenic member of the oral microbiome, where it may promote development of oral squamous cell carcinomas (Pignatelli et al., Microoganisms. (2023) 11:2358). It is therefore possible that bacteria in the esophagus are oncogenic or protective, and such bacteria will likely demonstrate cancer or healthy tissue specific presence patterns.

The most accessible data for studying the tumor microenvironment are short-read transcriptome (RNAseq) data. In addition to studying the presence of organisms, these data can provide insight into the complement of microbial proteins that are expressed in an environment (Ranjan et al., Microbial metatranscriptomics belowground. Singapore: Springer Singapore. (2021) p.1-36). However, RNAseq reads are typically very short, introducing several challenges to analysis of diverse bacterial species (Breitwieser et al., Brief Bioinform. (2019) 20:1125-36). For example, RNAseq reads in The Cancer Genome Atlas (TCGA) are typically 48 or 75 nucleotides. The length and abundance of microbial reads make de novo assembly of longer coding sequences extremely challenging (Breitwieser et al., Brief Bioinform. (2019) 20:1125-36; Celaj et al., Microbiome. (2014) 2:39). Methods for read identification without assembly, using alignment (Wood and Salzberg, Genome Biol. (2014) 15: R46) or other sequence search approaches, rely on databases of sequenced organisms. However, the size of microbial databases poses a computational challenge for such approaches, which are limited in precision by the short length of each sequence (Breitwieser et al., Brief Bioinform. (2019) 20:1125-36; Celaj et al., Microbiome. (2014) 2:39).

Despite these limitations, screening large volumes of cancer RNAseq reads, such as those included in TCGA, for sequences of likely microbial origin has been used to identify varied and complex bacterial populations of tumors (Robinson et al., Microbiome. (2017) 5:1-17; Nejman et al., Science. (2020) 368:973-80; Poore et al., Nature. (2020) 579:567-74). Comparisons between samples taken from tumors and nearby non-cancerous tissue have shed further light on the differences between tumor and adjacent microenvironments, revealing diverse microbial species with shifted prevalence in cancer (Dohlman et al., Cell Host Microbe. (2021) 29:281-.e5; Narunsky-Haziza et al., Cell. (2022) 185:3789-.e17). In a comparative study of several cancer types, ESCA had a high abundance of bacterial reads, consistent with other GI tract cancers, but among the lowest prevalence of fungal reads (Dohlman et al., Cell Host Microbe. (2021) 29:281-.e5). These studies have focused on data from only cancer patients in TCGA or similar datasets; however, tumor-adjacent tissues are not necessarily healthy (Aran et al., Nat Commun. (2017) 8:1077) and may not capture the full range of variation between healthy and cancer microbiota.

Thus, there is a need in the art for improved detection of reads of microbial origin in the tumor microenvironment. The present invention satisfies this unmet need.

### SUMMARY OF THE INVENTION

In some embodiments, the invention relates to a method of detecting one or more microbial populations or microbial gene expression in a sample, comprising the steps of: training a model to predict an origin of a nucleotide base-pair sequence; obtaining reads of transcriptome data of the sample; and using the model to determine the origin of the reads of the transcriptome data.

In some embodiments, the model is a convolutional neural network with at least one convolutional layers and at least one fully-connected layer.

In some embodiments, the model is trained on a set of human nucleotide base pair sequences, bacterial nucleotide base pair sequences, microbial nucleotide base pair sequences, or a combination thereof.

In some embodiments, the nucleotide base-pair sequence is a segmented nucleotide base-pair sequence.

In some embodiments, the step of training the model comprises the steps of: obtaining a training set of nucleotide base pair sequences comprising human nucleotide base pair sequences, bacterial nucleotide base pair sequences, microbial nucleotide base pair sequences, or a combination thereof; labeling the human nucleotide base pair sequences, bacterial nucleotide base pair sequences, or microbial nucleotide base pair sequences in the training set as a human sequence, a bacterial sequence, or a microbial sequence respectively; training the model to discriminate between human sequence, a bacterial sequence, or a microbial sequence with a first subset of the training set; and validating the model against a second non-overlapping subset of the training set by comparing a predicted origin of each nucleotide base pair sequences.

In some embodiments, the prediction comprises assigning a score to each nucleotide base pair sequence denoting the relative likelihood of the origin of the nucleotide base pair sequence.

In some embodiments, the method further comprises the step of assembling the reads determined to be of similar origin into longer sequences.

In some embodiments, the determined origin of reads is selected from one or more of the group consisting of: microbial, bacterial, viral, and human.

In some embodiments, the sample is a human tissue sample.

In some embodiments, the method further comprises the step of excluding all reads that map to a human genome.

In some embodiments, the reads are aligned to a database of known microbial sequences.

In some embodiments, the invention relates to a system for detecting one or more microbial populations or microbial gene expression in a sample comprising: a model for predicting an origin of a nucleotide base-pair sequence; reads of transcriptome data of the sample; a non-transitory computer readable medium with instructions stored thereon, which when executed by a processor perform steps comprising using the model to determine the origin of the reads of the transcriptome data.

In some embodiments, the model is a convolutional neural network with at least one convolutional layer and at least one fully-connected layer.

In some embodiments, the model is trained on a set of human nucleotide base pair sequences, bacterial nucleotide base pair sequences, microbial nucleotide base pair sequences, or a combination thereof.

In some embodiments, the nucleotide base-pair sequence is a segmented nucleotide base-pair sequence.

In some embodiments, the model is trained by: obtaining a training set of nucleotide base pair sequences comprising human nucleotide base pair sequences, bacterial nucleotide base pair sequences, microbial nucleotide base pair sequences, or a combination thereof; labeling the human nucleotide base pair sequences, bacterial nucleotide base pair sequences, or microbial nucleotide base pair sequences in the training set as a human sequence, a bacterial sequence, or a microbial sequence respectively; training the model to discriminate between human sequence, a bacterial sequence, or a microbial sequence with a first subset of the training set; and validating the model against a second non-overlapping subset of the training set by comparing a predicted origin of each nucleotide base pair sequences.

In some embodiments, the model assigns a score to each nucleotide base pair sequence denoting the relative likelihood of the origin of the nucleotide base pair sequence.

In some embodiments, the system further assembles reads determined to be of similar origin into longer sequences.

In some embodiments, the determined origin of reads is selected from one or more of the group consisting of: microbial, bacterial, viral, and human.

In some embodiments, the sample is a human tissue sample.

In some embodiments, the system further excludes all reads that map to a human genome.

In some embodiments, the system further aligns reads to a database of known microbial sequences.

In some embodiments, the invention relates to a method for diagnosing a subject as having, or being at risk for having, esophageal cancer in the subject comprising obtaining a biological sample from the subject and detecting one or more bacterial populations or microbial gene expression in the sample using to a system for detecting one or more microbial populations or microbial gene expression in a sample comprising: a model for predicting an origin of a nucleotide base-pair sequence; reads of transcriptome data of the sample; a non-transitory computer readable medium with instructions stored thereon, which when executed by a processor perform steps comprising using the model to determine the origin of the reads of the transcriptome data.

In some embodiments, the invention relates to a method of assessing a prognosis of a subject having esophageal cancer comprising obtaining a biological sample from the subject and detecting one or more bacterial populations or microbial gene expression in the sample using to a system for detecting one or more microbial populations or microbial gene expression in a sample comprising: a model for predicting an origin of a nucleotide base-pair sequence; reads of transcriptome data of the sample; a non-transitory computer readable medium with instructions stored thereon, which when executed by a processor perform steps comprising using the model to determine the origin of the reads of the transcriptome data.

In some embodiments, the method further comprises a step of administering a treatment.

In some embodiments, the invention relates to a method for diagnosing a subject as having, or being at risk for having, cancer comprising: obtaining a biological sample from the subject; measuring the abundance of at least one bacteria, at least one bacterial protein, at least one protein from the subject, or a combination thereof in the biological sample; and comparing the level of the at least one bacteria, at least one bacterial protein, or combination thereof in the biological sample to a comparator, wherein a differential level in the at least one bacteria, at least one bacterial protein, or combination thereof in the biological sample relative to the comparator indicates the subject has, or is at risk for having, cancer.

In some embodiments, the cancer is esophageal cancer.

In some embodiments, the at least one bacteria is one or more bacteria from a genera selected from the group consisting of: Cutibacterium, Sphigomonas, Fictibacillus, Corynebacterium, Bacillus, Gluconacetobacter, Peribacillus, Candidimonas, Burkholderia, Delfita, Halopseodomonas, Methylophilus, and Larkinella.

In some embodiments, a decrease in bacteria from the genera selected from the group consisting of Cutibacterium, Sphigomonas, Fictibacillus, and Corynebacterium relative to the comparator indicates the subject has, or is at risk for having, cancer.

In some embodiments, an increase in bacteria from the genera selected from the group consisting of Bacillus, Gluconacetobacter, Peribacillus, Candidimonas, Burkholderia, Delfita, Halopseodomonas, Methylophilus, and Larkinella relative to the comparator indicates the subject has, or is at risk for having, cancer.

In some embodiments, the at least one bacterial protein is one or more selected from the group consisting of: translation elongation factor EF-1 alpha, ferritin, NADHquinone oxidoreductase subunit H, a zincin-like metallopeptidase protein, DNA topoisomerase III, a transposase, a phage replicative protein, acyl-CoA dehydrogenase, LLM-class flavin dependent oxidoreductase, an ABC transporter component, a peptidase, an S49 peptidase, and a phosphatase.

In some embodiments, a decrease in bacterial protein selected from the group consisting of translation elongation factor EF-1 alpha, ferritin, NADHquinone oxidoreductase subunit H, a zincin-like metallopeptidase protein, DNA topoisomerase III, and a transposase relative to the comparator indicates the subject has, or is at risk for having, cancer.

In some embodiments, an increase in bacterial protein selected from the group consisting of a phage replicative protein, acyl-CoA dehydrogenase, LLM-class flavin dependent oxidoreductase, an ABC transporter component, a peptidase, an S49 peptidase, and a phosphatase relative to the comparator indicates the subject has, or is at risk for having, cancer.

In some embodiments, the invention relates to a method of assessing a prognosis of a subject having cancer comprising: obtaining a biological sample from the subject; measuring the abundance of at least one bacteria, at least one bacterial protein, at least one protein from the subject, or a combination thereof in the biological sample; and comparing the level of the at least one bacteria, at least one bacterial protein, or combination thereof in the biological sample to a comparator, wherein a differential level in the at least one bacteria, at least one bacterial protein, or combination thereof in the biological sample relative to the comparator indicates the prognosis of the subject having cancer.

In some embodiments, the cancer is esophageal cancer.

In some embodiments, the at least one protein from the subject is one or more selected from the group consisting of SAT1, SAT2, FTL, MAP11C3B2, MAP11C3B, and VDAC2.

In some embodiments, an increase in the at least one protein from the subject relative to the comparator indicates the subject has a poor prognosis.

In some embodiments, the at least one bacterial protein is one or more selected from the group consisting of: a phage protein, a ribosomal protein, an MFS transporter, a protein linked to mitochondrial function, and an iron-sulfur cluster protein.

In some embodiments, a decrease in a protein linked to mitochondrial function, an iron-sulfur protein, or a combination thereof, relative to the comparator indicates the subject has a poor prognosis.

In some embodiments, the protein linked to mitochondrial function is selected from the group consisting of pyruvate dehydrogenase, succinate dehydrogenase and aconitase.

In some embodiments, the iron-sulfur cluster protein is selected from the group consisting of aconitase, succinate dehydrogenase iron-sulfur, and iron-sulfur cluster assembly SufB.

In some embodiments, an increase in at least one bacterial protein selected from the group consisting of a phage protein, a ribosomal protein, an MFS transporter relative to the comparator indicates the subject has a poor prognosis.

In some embodiments, the biological sample is selected from the group consisting of: blood, serum, plasma, gastric secretions, pancreatic juice, a gastrointestinal biopsy sample, microdissected cells from an esophageal biopsy, esophageal cells sloughed into the gastrointestinal lumen, esophageal cells recovered from stool, a stool sample, and an esophageal tissue.

In some embodiments, the method further comprises a step of administering to the subject a therapeutic agent to treat or prevent cancer.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A through FIG. 1D depict data demonstrating the read-classification model architecture and performance. FIG. 1A depicts an overview of the model architecture. FIG. 1B depicts test-set one-versus-all precision recall curves for each class of sequence origin. FIG. 1C depicts test-set one-versus-all receiver-operating characteristic curves for each class. The AUCs are the areas under each curve. FIG. 1D depicts model scores for 1000 randomly-selected sequences from each class, plotted on the x+y+z=1 plane.

FIG. 2A through FIG. 2C depict data demonstrating bacterial genera over- and underabundant in esophogeal carcinoma vs. healthy tissues. FIG. 2A depicts a histogram of the numbers of district bacterial species detected in each ESCA (TCGA, red) and healthy (GTEx, blue) sample. FIG. 2B depicts A scatterplot of the abundance in ESCA and healthy esophagus of each bacterial genera; genera with sufficient representation and with significant differences are colored red if overabundant in ESCA and blue if underabundant in ESCA. Genera with 50 percentage-point differences in abundance are labeled. FIG. 2C depicts A 16S rRNA-based tree of bacterial genera with sufficient representation in ESCA or healthy esophagus. Genera that are significantly overabundant in ESCA are shown in red, and genera that are significantly underabundant in ESCA are shown in blue.

FIG. 3A through FIG. 3C depict data demonstrating microbial genes associated with progression free survival. FIG. 3A depicts Circle heatmaps showing the normalized proportion of samples positive for microbial genes (y-axis) from different bacteria (x-axis) in ESCA cancer (upper panel, in red) and normal esophagus (bottom panel, in blue). Proportions are normalized so the values in each column sum to 1, i.e., each (protein, genus) value indicates the proportion of samples positive for any of the proteins from that genus that are positive for the given protein. FIG. 3B depicts bar plots showing the overall proportion of each bacterial gene, from all species, in ESCA cancer (red) and normal esophagus (blue) samples. FIG. 3C depicts Kaplan Meier curves comparing the DSS between ESCA patients positive (red) and negative (blue) for each bacterial gene. The log-rank p-value is reported for significant associations with FDR-corrected q<0.05.

FIG. 4A through FIG. 4D depict host upregulated pathways in ESCA samples positive for FE-genes. FIG. 4A depicts a heatmap showing the gene expression (RSEM Z-score) of human genes upregulated in Fe-genes positive samples, belonging to four pathways significantly upregulated. FIG. 4B depicts boxplots comparing the average gene expression of genes in the four pathways between Fe-genes positive and negative samples. FIG. 4C depicts Kaplan Meier curves comparing the PFS between ESCA patients positive vs negative to any of the Fe-genes, and right panel FIG. 4D depicts the PFS between ESCA patients with high vs low average ferroptosis gene expression level (using the median as threshold).

FIG. 5 depicts representative experiments demonstrating the effect of random mutation on model performance. To understand the effect of including reads containing N's, as well as reads that were padded from 75 bp to 76 bp, on the pipeline, the performance of the classification model on reads was examined from the validation set with 0, 1, or 2 randomly-selected bases changed to a different nucleotide. Class one-versus-all AUPRCs are shown for 0-4 random mutations for each of bacterial, viral, and human simulated reads. With one mutation, class one-versus-all AUPRCs were reduced by 0.016 for human, 0.010 for bacteria, and 0.022 for virus. With two mutations, AUPRCs were reduced by 0.032, 0.021, and 0.045, respectively. This was assessed to be a relatively small impact in performance, especially as it is expected to correctly replace an N 25% of the time on actual reads. Therefore, RNAseq reads were included with at most one N in the pipeline as well as using the 76-basepair model on 75-bp TCGA reads rather than retraining a 75-bp model. Further mutations had a roughly linear increasing impact on performance, as shown.

FIG. 6 depicts example experiments comparing “seed” read score thresholds. The number of test-set simulated sequences that would be selected as a “seed,” in millions, based on the model scores and one of five possible thresholds. The first four thresholds describe a minimum value on either the bacterial or viral scores. The last threshold describes a maximum threshold on the human score. Reads that pass each threshold are categorized as correct pathogen (bacterial/viral reads whose bacterial/viral score is highest), opposite pathogen (bacterial/viral reads whose viral/bacterial score is highest), and human reads.

FIG. 7 depicts the number of genera detected with varying contig thresholds. The number of bacterial genera that are found in at least 10% of GTEx or TCGA esophageal samples, where “found” is defined as assigning a minimum of k reads to a sequence from that genus, for values of k between 1 and 10. Genera are grouped by whether they are significantly over-prevalent in GTEx samples (binomial pFDR <0.05), over-prevalent in TCGA samples, or not significant in either direction.

FIG. 8A through FIG. 8C depict host metabolic shift associated with microbial protein presence in ESCA samples. FIG. 8A depicts a heat map illustrating oxidative phosphorylation genes that are upregulated in ESCA samples positive for microbial proteins. FIG. 8B and FIG. 8C depict violin plots comparing the predicted flux (using genome scale metabolic modeling) in ATP generating reactions (FIG. 8B) and oxygen consuming reactions (FIG. 8A). The rank-sum p-values are reported.

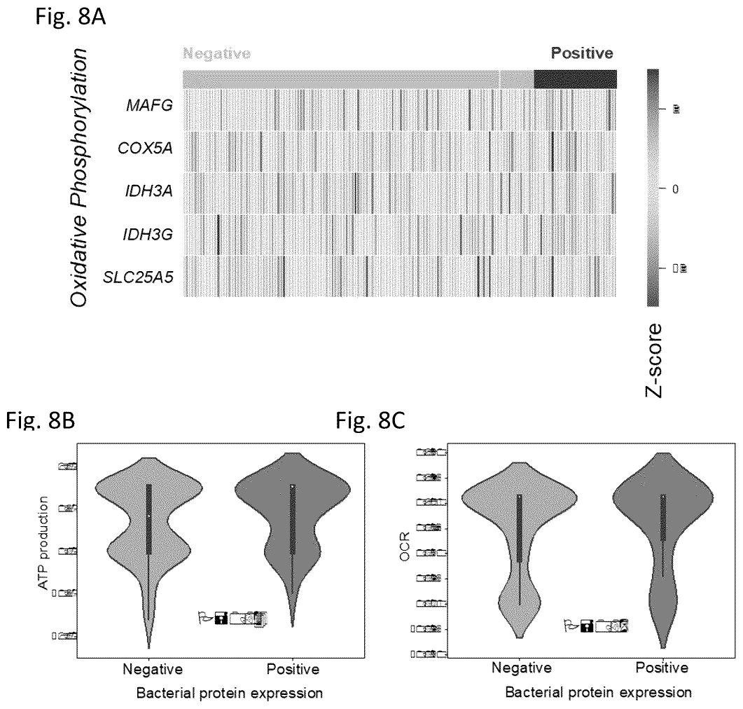

FIG. 9 depicts an exemplary method for detecting a microbial population or microbial gene expression in a sample.

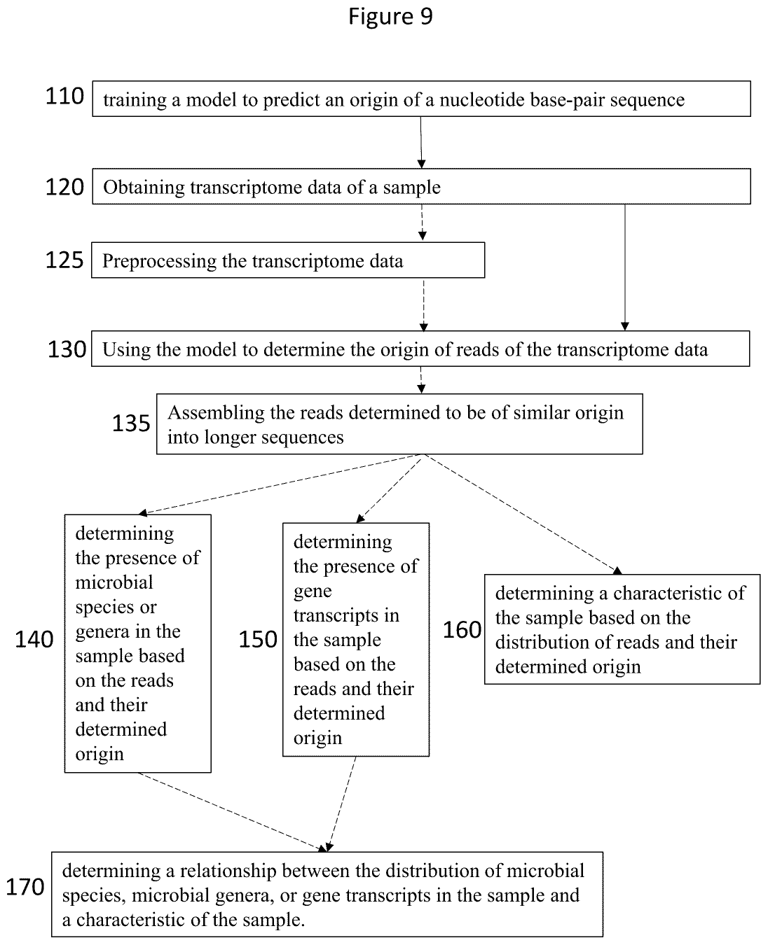

FIG. 10 depicts an exemplary computing device.

### DETAILED DESCRIPTION

The invention relates to a new tool to efficiently detect viral and bacterial expression in human tissues through RNAseq. The invention employs a neural network to predict reads of likely microbial origin, which are targeted for assembly into longer contigs, improving identification of microbial species and genes. In some embodiments, the invention is applied to perform a systematic comparison of bacterial expression in ESCA and healthy esophagi.

### Definitions

Unless defined otherwise, all technical and scientific terms used herein have the same meaning as commonly understood by one of ordinary skill in the art to which this invention belongs.

While the following terms are believed to be well understood by one of ordinary skill in the art, the following definitions are set forth to facilitate explanation of the presently disclosed subject matter.

As used herein, the term “a” or “an” can refer to one or more of that entity, i.e., can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is, by way of non-limiting examples, a human, a dog, a cat, a horse, or other domestic mammal.

The term “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid (e.g., microRNA, etc.) or a protein, or abundance of a microorganism, such as a bacteria, in a sample as compared to a control or reference level. For example, the quantity of a particular biomarker may be present at an elevated amount or at a decreased amount in samples of patients with a disease compared to a reference level. In one embodiment, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” includes determining the amount of something present, as well as determining whether it is present or absent.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between compared to a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between compared to a comparator.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, “treating a disease or disorder” means reducing the severity and/or frequency with which at least one sign or symptom of the disease or disorder is experienced by a patient.

The term “normobiosis” (also called “eubiosis” or “probiosis”) of oral biofilms refers to a microbiota composition with higher levels of beneficial bacteria and/or bacterial activity, while disease-associated species are present, but in a lower abundance.

Normobiosis includes more resilience to diseases, which means more resistance to disease drivers (i.e. a protective effect to any factor that can cause disease) and a quicker recovery from a perturbation caused by a disease driver.

The term “dysbiosis,” as used herein, refers to imbalances in quality, absolute quantity, or relative quantity of members of the microbiota of a subject, which is sometimes, but not necessarily, associated with the development or progression of a disease or disorder.

As used herein, the term “gastrointestinal tract” (“GI”) or “gut” refers to the entire alimentary canal, from the oral cavity to the rectum. The term encompasses the tube that extends from the mouth to the anus, in which the movement of muscles and release of hormones and enzymes digest food. The gastrointestinal tract starts with the mouth and proceeds to the esophagus, stomach, small intestine, large intestine, rectum and, finally, the anus.

The term “microbiota,” as used herein, refers to the population of microorganisms present within or upon a subject. The microbiota of a subject includes commensal microorganisms found in the absence of disease and may also include pathobionts and disease-causing microorganisms found in subjects with or without a disease or disorder.

As used herein, the term “microbiome” refers to the totality of microbes (bacteria, fungae, protists), their genetic elements (genomes) in a defined environment. In one embodiment, the microbiome is a gut microbiome (e.g., esophageal microbiome). The term “gut microbiome” as used herein can refer to the totality of microorganisms, bacteria, viruses, protozoa and fungi and their collective genetic material present in the gastrointestinal tract (GIT).

The term “gut microbe” as used herein can refer to any commensal or pathogenic microorganisms, bacteria, viruses, protozoa and fungi that colonize the gastrointestinal tract (GIT) or gut. The term “gut microbiota” as used herein can refer to the collection or population of microorganisms, bacteria, viruses, protozoa and fungi, commensal and pathogenic, residing in the GIT.

The terms “pathobiont” or “pathogenic microbe” are used interchangeably and refer to potentially disease- or disorder-causing members of the microbiota that are present in the microbiota of a non-diseased or a diseased subject, and which has the potential to contribute to the development or progression of a disease or disorder.

The term “beneficial microbe,” as used herein, refers to members of the microbiota that are present in the microbiota of a non-diseased or a diseased subject, and which has the potential to contribute to the reduction of the severity and/or frequency with which at least one sign or symptom of the disease or disorder is experienced by a subject having a disease or disorder.

“Isolated” means altered or removed from the natural state. For example, a microbe naturally present in its normal context in a living animal is not “isolated,” but the same microbe partially or completely separated from the coexisting materials of its natural context is “isolated.” An isolated microbe can exist in substantially purified form, or can exist in a non-native environment such as, for example, a gastrointestinal tract.

An “effective amount” or “therapeutically effective amount” of a compound is that amount of a compound which is sufficient to provide a beneficial effect to the subject to which the compound is administered.

A “therapeutic” treatment is a treatment administered to a subject who exhibits at least one sign or symptom of a disease or disorder, or is at risk of developing at least one sign or symptom of a disease or disorder, for the purpose of diminishing or eliminating those signs or symptoms, or reducing the likelihood of developing at least one sign or symptom of a disease or disorder.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, rectal, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

The term “regulating” or “modulating” as used herein can mean any method of altering the level or activity of a substrate (e.g., microbiome). Non-limiting examples of regulating with regard to a microbiome or microbiota further include affecting the microbiome or microbiota activity.

The term “regulator” or “modulator” refers to a molecule whose activity includes affecting the level or activity of a substrate (e.g., microbiome). A regulator can be direct or indirect. A regulator can function to activate or inhibit or otherwise modulate its substrate (e.g., microbiome).

The terms “silence”, “silencing”, “inhibit”, and “inhibition,” as used herein, means to reduce, suppress, diminish, or block an activity or function relative to a control value. For example, in one embodiment, the activity is suppressed or blocked by at least about 10% relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%.

As used herein, a “probiotic” refers live, non-pathogenic microorganisms, e.g., bacteria, which can confer health benefits to a host organism that contains an appropriate amount of the microorganism. In some embodiments, the host organism is a mammal. In some embodiments, the host organism is a human. Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012, Science 338, 120-123; Cuevas-Ramos et al., 2010, Proc. Natl. Acad. Sci. U.S.A. 107, 11537-11542; Nougayrède et al., 2006, Science 313, 848-851). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, a “prebiotic” refers to an ingredient that allows specific changes both in the composition and/or activity in the gastrointestinal microbiota that may (or may not) confer benefits upon the host. In some embodiments, a prebiotic can be a comestible food or beverage or ingredient thereof. Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

The phrase “biological sample” as used herein, is intended to include any sample comprising a cell, a tissue, feces, or a bodily fluid in which the presence of a microbe, nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, the term “microorganism,” or “microbe,” refers to a microscopic organism. In some embodiments, the term “microorganism” will be understood to include bacteria, fungi, protozoa (e.g., protozoan parasites), viruses (e.g., DNA viruses and/or RNA viruses), algae, archaea, phages, and/or helminths (e.g., multicellular eukaryotic parasites). In some embodiments, a microorganism is a single-celled organism and/or a colony of single-celled organisms. In some embodiments, a microorganism is eukaryotic or prokaryotic. In some embodiments, a microorganism is a pathogen (e.g., disease-causing), such as a human, animal, or plant-infective pathogen.

In some embodiments, as used herein, the term “model” refers to a machine learning model or algorithm. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep- and -wide sample-level classifier). In some embodiments, a model comprises 100 or more, 1000 or more, 10,000 or more, 100,000 or more or 1×10⁶ or more parameters.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “sequence read” or “read” refers to a sequence read from a portion of a nucleic acid sample. Typically, though not necessarily, a read represents a short sequence of contiguous base pairs in the sample. In some embodiments, a read is represented symbolically by the base pair sequence (in ATCG) of the sample portion. In some cases, a read is stored in a memory device and processed as appropriate to determine whether it matches a reference sequence or meets other criteria. In some instances, a read is obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and mapped to a chromosome or genomic region or gene.

In some embodiments, sequence reads are produced by any sequencing process described herein or known in the art. In some cases, reads are generated from one end of nucleic acid fragments (“single-end reads”) or from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. In some embodiments, sequence reads are obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the development of a method and system to identify the origin of a nucleotide sequence.

In some embodiments, the invention relates to a method 100 for detecting a microbial population or microbial gene expression in a sample. In some embodiments, the method includes the steps of 110 training a model to predict an origin of a nucleotide base-pair sequence, 120 obtaining transcriptome data of a sample, and 130 using the model to determine the origin of reads of the transcriptome data. In some embodiments, the sample is a tissue sample. In some embodiments, the tissue sample is a human tissue sample. In some embodiments, the origin of the nucleotide base-pair sequence is a microbial origin. In some embodiments, the origin of the nucleotide base-pair sequence is a human origin.

In some embodiments, the method further includes the step of 125 preprocessing the transcriptome data. In some embodiments of the method, step 125 is performed before step 130. In some embodiments, the method further includes the step of 135 assembling the reads determined to be of a similar origin into longer sequences. In some embodiments, the method further includes the step of 140 determining the presence of microbial species or genera in the sample based on the reads and their determined origin. In some embodiments, the method further includes the step of 150 determining the presence of gene transcripts in the sample based on the reads and their determined origin. In some embodiments, the gene transcript is of a microbial gene, a human gene, or a combination thereof. In some embodiments, the method further includes the step of 160 determining a characteristic of the tissue sample based on the distribution of reads and their determined origin. In some embodiments, the method further includes the step of 170 determining a relationship between the distribution of microbial species, microbial genera, and/or gene transcripts in the sample and a characteristic of the sample.

In some embodiments, the method 100 for detecting a microbial population or microbial gene expression in a sample includes the step of 110 training a model to predict an origin of a nucleotide base-pair sequences. In some embodiments, the sample is a tissue sample. In some embodiments, the tissue sample is a human tissue sample. In some embodiments, the origin of the nucleotide base-pair sequence is a microbial origin. In some embodiments, the origin of the nucleotide base-pair sequence is a human origin. The model may be trained with nucleotide base-pair sequences obtained from human and/or microbial transcriptome data. The transcriptome data may be derived from any source or database. In some embodiments, the transcriptome data used to train the model may simulate reads obtained from RNA sequencing. In some embodiments, the transcriptome data used to train the model may be reads obtained from RNA sequencing. In some embodiments, nucleotide base-pair sequences of human origin, viral origin, and bacterial origin are used to train the model. In some embodiments, nucleotide base pair sequences, bacterial nucleotide base pair sequences, or microbial nucleotide base pair sequences are labeled as a human sequence, a bacterial sequence, or a microbial sequence. In some embodiments, an equal or approximately equal number of human origin base-pair sequences, viral origin base-pair sequences, and bacterial origin base-pair sequences are used to train the model. Using an equal or approximately equal number of base-pair sequences from all origins may allow for balanced training of the model. In some embodiments, a different number of human origin base-pair sequences, viral origin base-pair sequences, and bacterial origin base-pair sequences are used to train the model. In some embodiments, any transcriptome data may be segmented into base pair sequences of any length before being used to train the model. In some embodiments, nucleotide base-pair sequences used to train the model is 1 base pair long, 2 base pairs long, 3 base pairs long, 4 base pairs long, 5 base pairs long, 6 base pairs long, 7 base pairs long, 8 base pairs long, 9 base pairs long, or 10 or more base pairs long. In some embodiments, the nucleotide base-pair sequences used to train the model are about 10 to about 20 base pairs long. In some embodiments, the nucleotide base-pair sequences used to train the model is about 20 to about 30 base pairs long. In some embodiments, the nucleotide base-pair sequences used to train the model are about 30 to about 40 base pairs long. In some embodiments, the nucleotide base-pair sequences used to train the model are about 40 to about 50 base pairs long. In some embodiments, the nucleotide base-pair sequences used to train the model are about 50 to about 100 base pairs long. In some embodiments, the nucleotide base-pair sequences used to train the model are about 100 to about 200 base pairs long. In some embodiments, the nucleotide base-pair sequences used to train the model are about 200 to about 300 base pairs long. In some embodiments, the nucleotide base-pair sequences used to train the model are about 300 to about 400 base pairs long. In some embodiments, the nucleotide base-pair sequences used to train the model are about 400 to about 500 base pairs long. In some embodiments, the nucleotide base-pair sequences used to train the model are greater than about 500 base pairs long. In some embodiments, the nucleotide base-pair sequences used to train the model are or are about 76 base pairs long. In some embodiments, the nucleotide base-pair sequences used to train the model are or are about 75 base pairs long. In some embodiments, the nucleotide base-pair sequences used to train the model are or are about 48 base pairs long. In some embodiments, the length of nucleotide base-pair sequences used to train the model is chosen to match read lengths of RNA sequencing data. In some embodiments, the length of nucleotide base-pair sequences used to train the model is chosen to match read lengths of any RNA sequencing data that one desires the model to predict the origin of. In some embodiments, nucleotide base pair sequences of all origins may be divided randomly into a model training set, a model validation set, and a model testing set.

In some embodiments, the segmentation of transcriptome data is random or systematic. In some embodiments, the segmentation of transcriptome data is performed using any filtering method. In some embodiments, the segmentation of transcriptome data is performed by segmenting with any stride length. Stride lengths may be chosen for generating balanced data among transcriptome data from different origins. For example, smaller stride lengths may be chosen for some origins to generate more base-pair sequences for training and greater stride lengths may be chosen for some origins to generate less base-pair sequences such that balance among read origins is achieved. In some embodiments, nucleotide base-pair sequences used to train the model are all the same length or a similar length. The chosen stride length may be any stride length, for example stride length 2, 3, 4, 5, 6, 7, 8, 9, 10, or more. In some embodiments, nucleotide base-pair sequences used to train the model are all the different length. In some embodiments, nucleotide base-pair sequences used to train the model are a combination of same, similar, and/or different length. In some embodiments, segments may contain unspecified nucleotides. In some embodiments, segments containing any unspecified nucleotides, also referred to as N's, are excluded from any model training, validation, or testing.

Human origin nucleotide base-pair sequences for model training may be derived from any source or database. In some embodiments, a reference human transcriptome may be used to generate training data, for example the human hg19 reference transcriptome obtained from NCBI (Sayers et al. Nucleic Acids Research 2021). Viral origin nucleotide base-pair sequences for model training are derived from any source or database. In some embodiments, sequences may be derived from databases of any number of different viral species. In some embodiments, viral origin base-pair sequences may be obtained from any database or databases of transcripts derived from diverse viruses of placental mammals, for example the Virus Variation Resource (Hatcher et al. Nucleic Acids Research 2017). Bacterial origin base-pair sequences for model training may be derived from any source or database. In some embodiments, the database may include representative bacterial genomes from different bacterial species or genera. For example, a database may be curated to include the same number of representative bacterial genomes for any number of bacterial genera. For example, a curated database of bacterial genomes may be used containing one representative per genus (Auslander et al. Nucleic Acids Research 2020). Genome databases may be converted to transcriptome databases using any method.

In some embodiments, the model is a neural network. Exemplary suitable neural networks are described in U.S. patent application Ser. No. 18/392,646 and is incorporated by reference herein in its entirety.

In some embodiments, the model is a small convolutional neural network. In some embodiments, the model is a small convolutional neural network with any number of convolutional layers and any number of fully connected layers. For example, the model may be a small convolutional neural network with two convolutional layers and one fully connected layer.

In some embodiments, the model includes any number of embedding layers (e.g. 1, 2, 3, 4, 5, 6, 7, 8, 9, or 10 or more). In some embodiments, the model includes 1 embedding layer. In some embodiments, the model includes any number of convolutional layers (e.g. 1, 2, 3, 4, 5, 6, 7, 8, 9, or 10 or more), where the respective parameters, or weights, for each convolutional layer are filters. In some embodiments, the model includes two 1D convolutional layers. In some embodiments, each convolutional layer comprises any number of filters (e.g. 1, 2, 3, 4, 5, 6, 7, 8, 9, or 10 or more). Each filter has a corresponding height and width. In some embodiments, each convolutional layer comprises 64 filters. Each filter may have any width (e.g. 1, 2, 3, 4, 5, 6, 7, 8, 9, or 10 or more). In some embodiments, each filter has a width of 64. In some embodiments, each filter has a width of 64 and padding with zeros. In some embodiments, the model includes any number of fully connected layers (e.g. 1, 2, 3, 4, 5, 6, 7, 8, 9, or 10 or more) with any number of units (e.g. 1, 2, 3, 4, 5, 6, 7, 8, 9, or 10 or more). In some embodiments, the model includes one fully connected layer. In some embodiments, fully connected layers of the model include any number of units. In some embodiments, the model includes one fully connected layer with 64 units. In some embodiments, the units of the fully connected layer includes 64 units. In some embodiments, the units of the fully connected layer include any activation function, for example ReLU activation. In some embodiments, the model includes an output layer with any activation function, for example SoftMax activation. Any learning rate or normalization may be used in the model. For example, the learning rate may be set to 0.0001 and L2 normalization with weight 0.01 may be used.

The model may be trained using any method. In some embodiments, the model is trained using TensorFlow 2.8. The model may be trained for any number of epochs (e.g. 1, 2, 3, 4, 5, 6, 7, 8, 9, or 10 or more). In some embodiments, the model is trained for 100 epochs. The model may be trained on any subset of the training dataset. The subset of the training dataset may be randomly selected.

In some embodiments, the method comprises obtaining a training set of nucleotide base pair sequences comprising human nucleotide base pair sequences, bacterial nucleotide base pair sequences, microbial nucleotide base pair sequences, or a combination thereof. In some embodiments, the method comprises labeling the human nucleotide base pair sequences, bacterial nucleotide base pair sequences, or microbial nucleotide base pair sequences in the training set as a human sequence, a bacterial sequence, or a microbial sequence respectively. In some embodiments, the method comprises training the model to discriminate between human sequence, a bacterial sequence, or a microbial sequence with a first subset of the training set. In some embodiments, the method comprises validating the model against a second non-overlapping subset of the training set by comparing a predicted origin of each nucleotide base pair sequences.

Any parameter, including hyper-parameters, may be tuned over the number of convolutional layers and units, the number of fully connected layers and units, the width of the convolutions, the width of the max pool, the learning rate, and the dropout throughout model training. Models of different parameters may be compared by any method, for example models may be compared based on validation-set one-versus-all area under the precision-recall curve (AUPRC).

In some embodiments, the method includes the step 120 of obtaining transcriptome data. In some embodiments, the transcriptome data is transcriptome data of at least one animal sample. In some embodiments, the animal is a mammal. In some embodiments, the animal is a human. In some embodiments, the sample is a tissue sample. In some embodiments, the sample is a human tissue sample. Transcriptome data of at least one animal sample may be obtained using any method or from any source. For example, transcriptome data may be obtained from The Cancer Genome Atlas (TCGA) or The Genotype Tissue Expression Project (GTEx) (Cancer Genome Atlas Research Network, et al. Nature 2017, Lonsdale et al. Nat Genet. 2013). The transcriptome data obtained of the at least one animal sample may be of the same type of data used to train the model. The transcriptome data obtained of the at least one animal sample may have aspects that are similar to the data used to train the model, for example any characteristic of read length. The transcriptome data of the at least one animal sample may be RNA sequencing data, for example short-read RNAseq data. The transcriptome data of the at least one animal sample may be obtained from any database or other resource. The transcriptome data of the at least one animal sample may be obtained by collecting a human tissue sample, collecting nucleic acid material from the sample, and performing any sequencing protocol.

The transcriptome data of the at least one animal sample may be a tissue sample from a human. For example, the transcriptome data may be of any tissue of any control subject or any subject that a has any disease, any condition, any genetic background, or any other trait. The transcriptome data of human tissue samples may be of a cancerous tissue or a tumor. The transcriptome data of human tissue samples may be of a control tissue or any non-cancerous tissue. Transcriptome data may be obtained from any number of human subjects or tissue types for comparison purposes (e.g. diseased state vs control). In some embodiments, the transcriptome data of human tissue is obtained from esophageal tissue, gastrointestinal tissue, intestinal tissue, colon tissue, rectal tissue, any tissue of the gastrointestinal tract, oral tissue, or any tissue that may have an associated microbiome. In some embodiments, the transcriptome data is obtained from a diseased tissue and a control tissue of the same tissue type. In some embodiments, the transcriptome data is obtained from a cancerous portion of a tissue and a nearby portion of a tissue that is non-cancerous. In some embodiments, the transcriptome data of at least one human tissue sample is of a patient. The patient may have any disease or condition, may be currently being diagnosed for any disease or condition, may be undergoing treatment for any disease or condition, or may be recovering from any disease or condition.

In some embodiments, the transcriptome data may be altered or preprocessed before using the model. In some embodiments, any reads of the transcriptome data that map to the human genome are removed from the dataset before the model is used to determine the likely origin of reads of the transcriptome data. Any human reference genome may be used to map reads of the transcriptome data to the human genome, for example the hg19 reference genome. In some embodiments, any reads of the transcriptome data with one or more unknown nucleotides, also referred to as N's, may be removed. In some embodiments, for any reads of the transcriptome data with one or more unknown nucleotides, also referred to as N's, N's may be replaced with a random nucleotide. In some embodiments, a decision to remove reads or replace N's may be made based on the number of unknown nucleotides. For example, for reads with a low number of unknown nucleotides, N's may be replaced with a random nucleotide and reads with a high number unknown nucleotides may be removed entirely. In some examples, N's are replaced by a random nucleotide for reads with only 1 or 2 unknown nucleotides and reads with more than 1 or 2 unknown nucleotides are removed. In some embodiments, reads may be altered to match the base pair length of the base pair sequences that were used to train the model. In some examples, any number of random nucleotides may be added to 3′ or 5′ ends of reads that are shorter than the read length of reads used to train the model.

In some embodiments, the method 100 includes the step of using the model to determine the origin of reads of the transcriptome data. In some embodiments, the model is used to determine if a read is of or is likely to be of human origin or microbial origin. In some embodiments, the model is used to determine if a read is of or is likely to be of human origin, bacterial origin, or viral origin. In some embodiments, the model assigns scores to each read that reflects the likelihood of each read to be of a specific origin. For example, the model may assign a human origin score and a microbial origin score to each read of the transcriptome data. In some examples, the model may assign a human origin score, a bacterial origin score, and viral origin score to each read of the transcriptome data. In some embodiments, an origin score is between the range of 0.00 and 1.00. In some embodiments, scores nearer to one end of the range represent a high likelihood of a read being of that origin and scores nearer to the opposite end of the range represent a low likelihood of a read being of that origin.

In some embodiments, after scores are assigned to each read by using the model, the reads are assembled into larger sequences. Assembling the reads into larger sequences may include combining individual reads that are likely to be from the same transcript such that larger sequences may be generated from shorter reads. In some embodiments, a threshold score is used to identify reads of likely microbial origin. For example, a threshold bacterial origin score and/or a threshold viral origin score may be used to identify reads of likely microbial origin. In some embodiments, reads may be sorted based on bacterial origin score and/or viral origin score to identify the likeliest reads to be of microbial origin, bacterial origin, and/or viral origin.

In some embodiments of the method, reads identified to be of likely microbial origin are assembled. Any assembly tool may be used to assemble longer sequences based on individual reads. Exemplary methods for assembling reads into longer sequences, and specifically assembling reads that have been identified to likely be of a particular origin (e.g. microbial, bacterial, or viral), are described in U.S. patent application Ser. No. 18/392,646. In some examples, the reads determined most likely to be of microbial origin, bacterial origin, or viral origin are used as seed reads. In some embodiments, reads may be sorted based on bacterial origin score and/or viral origin score to identify the likeliest reads to be of microbial origin, bacterial origin, and/or viral origin. The reads likeliest to be of bacterial origin may be used as seed reads. In some embodiments, the read with highest bacterial origin may be used as the first seed read, the read with the second highest bacterial origin score may be used as the second seed read and so on. Any portion of a seed read sequence, for example the sequence of either terminal end of the read, may be searched in all other reads. The searched portion may be or may be about any number of nucleotides long, for example 24 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, or 40 nucleotides.

If a portion of any other read matches the sequence of the seed read. The seed read sequence may be extended by using the sequence of the other read. In some embodiments, matching reads may be removed from the data after the seed read has been extended. In some embodiments, any reads that are wholly contained within the seed read may be removed. In cases in which a seed read or any other read contains unknown nucleotides or N's, N's may be considered to be a match to any nucleotide. In some embodiments, N's in a seed read that match to any other read may be replaced with a matching nucleotide. After all other sequences are searched and the seed read sequence appropriately extended, the next seed read may be searched and the process for extending a seed read repeated. This process may be repeated for all seed reads to complete the assembly process.

In some embodiments, the method includes the step of identifying the presence of microbial species in the sample based on the reads determined to be of microbial origin. In some embodiments, reads, or assembled reads, of the transcriptome data classified to be of or likely be of microbial origin, bacterial origin, or viral origin are compared to any database of nucleotide sequences to determine a microbial species from which they are derived. For example, blastn may be used to compare the reads or assemble reads to a curated database of microbial nucleotide sequences (Altschul et al. J Mol Biol. 1990). Any databases or curated databases may be used including NCBI representative bacterial genomes, any databases for reference human viruses, and/or any databases of novel or non-human viruses. In some embodiments, a read may be assigned to a species, or a genera. In some embodiments, a read may be assigned to the species or genera of the top hit when using any comparison tool for example BLAST. In some embodiments, a microbial species or genera may be determined to be present in a sample if at least one, two, 3, 4, 5, 6, 7, 8, 9, 10, or any number of reads is assigned to the microbial species or genera.

In some embodiments, the method includes the step of determining the presence of gene transcripts in the sample. In some embodiments, reads or assembled reads, determined to be of likely microbial origin are mapped to microbial genes. In some embodiments, the reads are mapped using any database of sequences including any microbial sequence database, for example RefSeq non-redundant microbial sequence database. Reads, or assembled reads, may be mapped using the aid of any tool, software, or program, for example blastx.

In some embodiments, the method includes the step of determining a characteristic of the tissue sample based on the distribution of reads of microbial origin and human origin. In some embodiments, the determination of a characteristic may be based on the microbial species and/or genera determined to be present in the sample, bacterial species and/or genera determined to be present in the sample, viral species and/or genera determined to be present in the sample, microbial gene transcripts determined to be present in the sample, bacterial gene transcripts determined to be present in the sample, viral gene transcripts determined to be present in the sample, human gene transcripts determined to be present in the sample, the gene expression levels of human genes in the sample, or any combination thereof.

The characteristic of the tissue sample may be a characteristic of the subject from the tissue sample was obtained. The characteristic may be the presence or absence of a disease, condition, genetic profile. The characteristic may be the presence or absence of any cancer including esophageal carcinoma or cancer of any tissue associated with a microbiome. The characteristic may be the progression or severity of a disease. The characteristic may be the response of a tissue, including a diseased tissue, to any treatment protocol. In some embodiments, the characteristic is a prognosis of a subject. The characteristic may be the risk of developing any disease or condition including esophageal cancer or cancer of any tissue associated with a microbiome. In some embodiments, the characteristic is determined based on the presence or absence of a subset of microbial genera or microbial transcripts.

In some embodiments, the method includes the step of determining a relationship between the distribution of microbial species, microbial genera, microbial transcripts, human transcripts, or combinations thereof in the sample and a characteristic of the at least one human tissue sample. Any statistical method or technique may be used to determine a correlation or relationship. For example, any number of transcriptome data from control tissues or tissues with any characteristic may be included in the method and the distribution of microbial species, microbial genera, microbial transcripts, human transcripts, or combinations thereof in the samples compared.

### Computer Systems and Methods

In some embodiments of the present invention, software or code for executing any number of the bioinformatic analysis required for execution of the methods of the invention may be stored on a non-transitory computer-readable medium, wherein the software performs some or all of the steps of the present invention when executed on a processor.

Embodiments of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

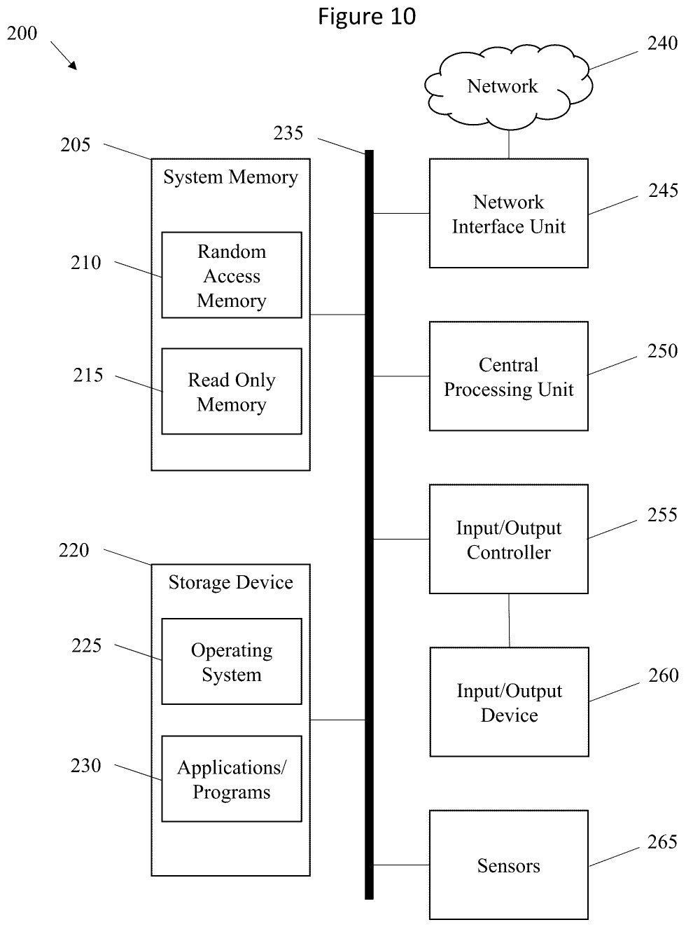

FIG. 10 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 10 depicts an illustrative computer architecture for a computer 200 for practicing the various embodiments of the invention. The computer architecture shown in FIG. 10 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 150. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device. Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like. According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 for monitoring the environment surrounding and the environment internal to the computer 200. These sensors 265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### Sample

The technology relates to the analysis of any sample associated with an esophageal disorder (e.g., BE, BED, BE-LGD, BE-HGD, EAC). For example, in some embodiments the sample comprises a tissue and/or biological fluid obtained from a patient. In some embodiments, the sample comprises esophageal tissue. In some embodiments, the sample comprises esophageal tissue obtained through whole esophageal swabbing or brushing. In some embodiments, the sample comprises a secretion. In some embodiments, the sample comprises blood, serum, plasma, gastric secretions, pancreatic juice, a gastrointestinal biopsy sample, microdissected cells from an esophageal biopsy, esophageal cells sloughed into the gastrointestinal lumen, and/or esophageal cells recovered from stool. In some embodiments, the subject is human. These samples may originate from the upper gastrointestinal tract, the lower gastrointestinal tract, or comprise cells, tissues, and/or secretions from both the upper gastrointestinal tract and the lower gastrointestinal tract. The sample may include cells, secretions, or tissues from the liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva. In some embodiments, the sample is a stool sample.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. In some embodiments, the sample is obtained through esophageal swabbing or brushing or use of a sponge capsule device.

### Method of Diagnosing Esophageal Cancer

The present invention further relates, in part, to a method of diagnosing a subject as having, or being at risk for having, esophageal cancer in a subject in need thereof. In some embodiments, the present invention relates, in part, to a method of detecting Barrett's Esophagus.

Barrett's Esophagus is a precursor lesion for most esophageal adenocarcinomas which is a malignancy with rapidly rising incidence and persistently poor outcomes. Early detection of esophageal adenocarcinoma has been shown to be associated with earlier stage and increased survival. Early detection of Barrett's Esophagus may enable placement of patients into surveillance programs which may allow detection of neoplastic progression at an earlier stage amenable to endoscopic or surgical therapy with improved outcomes. Screening for Barrett's Esophagus and esophageal adenocarcinoma has been hampered by the lack of a widely applicable tool, as well as the lack of a biomarker which can be combined with a screening tool. Acceptability and feasibility of screening by endoscopic and novel non-endoscopic methods has been demonstrated in the population. Non-endoscopic screening methods, such as by swallowed cytology brush or stool DNA testing, offer potential cost-effective alternatives to endoscopy for identification of Barrett's Esophagus in the general population. More recently, it has also shown that several aberrantly methylated genes could serve as highly discriminant markers for Barrett's Esophagus. Indeed, a study performed on archived frozen esophageal biopsies in patients with and without Barrett's revealed that a panel of tumor-associated genes was potentially useful to discriminate between Barrett's Esophagus and squamous mucosa. (see, e.g., Yang Wu, et al, DDW Abstract 2011).

Dysplasia is known to be distributed in a patchy manner in Barrett's esophagus, leading to “sampling error” on routine endoscopic surveillance as performed by four quadrant biopsies. It is known that conventional endoscopic surveillance with biopsies samples less than 10% of the BE segment. Compliance of endoscopists with conventional surveillance is known to be poor. While newer endoscopic techniques have been shown to improve the yield of dysplasia detection in studies performed in tertiary care centers, their applicability in the community remains uncertain. Methods which sample a larger mucosal surface area, such as swabbing or brushing, are likely to increase the yield of dysplasia and neoplasia, particularly if combined with molecular markers of dysplasia/neoplasia. This may ultimately allow non-biopsy (via swabbing or brushing) or non-endoscopic surveillance of BE subjects with potential substantial cost savings.

Accordingly, provided herein is technology for esophageal disorder screening and particularly, but not exclusively, to methods, compositions, and related uses for detecting the presence of esophageal disorders (e.g., Barrett's esophagus, Barrett's esophageal dysplasia, etc.). In addition, the technology provides methods, compositions and related uses for distinguishing between Barrett's esophagus and Barrett's esophageal dysplasia, and between Barrett's esophageal low-grade dysplasia, Barrett's esophageal high-grade dysplasia, and esophageal adenocarcinoma within samples obtained through endoscopic brushing or nonendoscopic whole esophageal brushing or swabbing using a tethered device (e.g. such as a capsule sponge, balloon, or other device).

In one aspect, the present invention provides a method of diagnosing a subject as having, or being at risk for having, esophageal cancer in a subject in need thereof, the method comprising obtaining a biological sample from the subject; measuring the abundance of at least one bacteria, at least one bacterial protein, at least one protein from the subject, or a combination thereof.

In some embodiments, the at least one bacteria is one or more bacteria from a genera selected from the group consisting of: Cutibacterium, Sphigomonas, Fictibacillus, Corynebacterium, Bacillus, Gluconacetobacter, Peribacillus, Candidimonas, Burkholderia, Delfita, Halopseodomonas, Methylophilus, and Larkinella.

Techniques to detect, identify, and/or analyze microorganisms are known in the art. Non-limiting examples include but are not limited to plating microorganisms, such as bacteria, on different media types. Another method involves differential staining of microorganisms, such as bacteria, with different chemicals such as Gram staining. A third method involves antibody staining to look for species-identifying proteins, for example, by ELISA detection protocols. A fourth method involves metagenomic sequencing, a variant of high-throughput sequencing which blasts reads to all known samples.

In some embodiments, the sample is in a liquid culture or suspended in a liquid culture. In some embodiments, the sample is in a liquid culture or suspended in a liquid culture for detection of the microorganism or measuring the abundance of the microorganism. In one embodiment, nucleic acid from a liquid culture comprising the microorganism, such as the bacteria, may be isolated and analyzed by any suitable technique to identify the microorganism. Exemplary methods for analysis of nucleic acids include, but are not limited to, amplification techniques, such as PCR and RT-PCR (including quantitative variants), and hybridization techniques, such as in situ hybridization, microarrays, and blots. In one embodiment, the nucleic acid may be analyzed to identify signature sequences from the microorganism of interest. The nucleic acid may be analyzed by PCR using primers that anneal, allow amplification, specifically to a signature nucleic acid sequence that occurs in the target microorganism.

The nucleic acid may be analyzed by PCR using primers that anneal specifically to a signature nucleic acid sequence that occurs in the target microorganism. The primers may anneal specifically to the signature nucleic acid sequence and/or may allow amplification of the specific signature nucleic acid. To increase the specificity more than one, more than two, more than three, more than four, more than five, more than six, more seven or more than eight signature sequences may be considered for the target microorganism to be detected. In one embodiment, at least 1, 2, 3, 4, 5, 6, 7, 8, 9, 10, 15, 20, 25, 30, 35, 40, 45, 50, 55, 60, 65, 70, 75, 80, 85, 90, 95, 100, or more than 100 signature species for at least one microorganism are evaluated in a single assay. Exemplary assays that can be used to evaluate multiple signature sequences, include, but are not limited to, microarrays, and q-PCR.

In one embodiment, the liquid culture comprising the microorganism is analyzed by sequencing. The nucleic acid sequence may be analyzed by sequencing at least a portion of the genomic DNA or RNA. Methods for performing whole or partial genome sequencing are known in the art and include, but are not limited to, exome sequencing, whole genome sequencing, and 16S rRNA sequencing. In various embodiments, sequencing may be done through Sanger sequencing, or through high-throughput next-generation sequencing techniques (e.g., using an Illumina based Hi-Seq, or Mi-Seq or Life Technologies PGM based sequencing platform).

In some embodiments, the abundance of a plurality of bacterial species from the genera selected from the group consisting of Cutibacterium, Sphigomonas, Fictibacillus, Corynebacterium, Bacillus, Gluconacetobacter, Peribacillus, Candidimonas, Burkholderia, Delfita, Halopseodomonas, Methylophilus, and Larkinella is measured.

In one embodiment, the method further comprises comparing the abundance of the at least one bacteria in the biological sample to the abundance of the same at least one bacteria in a comparator.

In some embodiments, a decrease in bacteria from the genera selected from the group consisting of Cutibacterium, Sphigomonas, Fictibacillus, and Corynebacterium relative to the comparator indicates the subject has, or is at risk for having, esophageal cancer. In some embodiments, an increase in bacteria from the genera selected from the group consisting of Bacillus, Gluconacetobacter, Peribacillus, Candidimonas, Burkholderia, Delfita, Halopseodomonas, Methylophilus, and Larkinella relative to the comparator indicates the subject has, or is at risk for having, esophageal cancer.

In some embodiments, an increase in bacteria from the genera selected from the group consisting of Cutibacterium, Sphigomonas, Fictibacillus, and Corynebacterium relative to the comparator indicates the subject does not have, or is not at risk for having, esophageal cancer. In some embodiments, an decrease in bacteria from the genera selected from the group consisting of Bacillus, Gluconacetobacter, Peribacillus, Candidimonas, Burkholderia, Delfita, Halopseodomonas, Methylophilus, and Larkinella relative to the comparator indicates the subject does not have, or is not at risk for having, esophageal cancer.

In some embodiments, the at least one bacterial protein is one or more selected from the group consisting of: translation elongation factor EF-1 alpha, ferritin, NADHquinone oxidoreductase subunit H, a zincin-like metallopeptidase protein, DNA topoisomerase III, a transposase, a phage replicative protein, acyl-CoA dehydrogenase, LLM-class flavin dependent oxidoreductase, an ABC transporter component, a peptidase, an S49 peptidase, and a phosphatase.

Methods for detecting a reduced expression or activity of one or more proteins comprise any method that interrogates a gene or its products at either the nucleic acid or protein level. Such methods are well known in the art and include, but are not limited to, nucleic acid hybridization techniques, nucleic acid reverse transcription methods, and nucleic acid amplification methods, western blots, northern blots, southern blots, ELISA, immunoprecipitation, immunofluorescence, flow cytometry, immunocytochemistry. In particular embodiments, disrupted gene transcription is detected on a protein level using, for example, antibodies that are directed against specific proteins. These antibodies can be used in various methods such as Western blot, ELISA, immunoprecipitation, flow cytometry, or immunocytochemistry techniques. In some embodiments, the at least one bacterial protein is one or more selected from the group consisting of: translation elongation factor EF-1 alpha, ferritin, NADHquinone oxidoreductase subunit H, a zincin-like metallopeptidase protein, DNA topoisomerase III, a transposase, a phage replicative protein, acyl-CoA dehydrogenase, LLM-class flavin dependent oxidoreductase, an ABC transporter component, a peptidase, an S49 peptidase, and a phosphatase.

In some embodiments, a decrease in bacterial protein selected from the group consisting of translation elongation factor EF-1 alpha, ferritin, NADHquinone oxidoreductase subunit H, a zincin-like metallopeptidase protein, DNA topoisomerase III, and a transposase relative to the comparator indicates the subject has, or is at risk for having, esophageal cancer. In some embodiments, an increase in bacterial protein selected from the group consisting of a phage replicative protein, acyl-CoA dehydrogenase, LLM-class flavin dependent oxidoreductase, an ABC transporter component, a peptidase, an S49 peptidase, and a phosphatase relative to the comparator indicates the subject has, or is at risk for having, esophageal cancer.

In some embodiments, an increase in bacterial protein selected from the group consisting of translation elongation factor EF-1 alpha, ferritin, NADHquinone oxidoreductase subunit H, a zincin-like metallopeptidase protein, DNA topoisomerase III, and a transposase relative to the comparator indicates the subject does not have, or is not at risk for having, esophageal cancer. In some embodiments, a decrease in bacterial protein selected from the group consisting of a phage replicative protein, acyl-CoA dehydrogenase, LLM-class flavin dependent oxidoreductase, an ABC transporter component, a peptidase, an S49 peptidase, and a phosphatase relative to the comparator indicates the subject does not have, or is not at risk for having, esophageal cancer. Information obtained from the methods of the invention described herein can be used alone, or in combination with other information (e.g., age, family history, disease status, disease history, vital signs, blood chemistry, PSA level, Gleason score, primary tumor staging, lymph node staging, metastasis staging, expression of other gene signatures relevant to outcomes of a disease or disorder, such as autoimmune disease or disorder, cancer, inflammatory disease or disorder, metabolic disease or disorder, neurodegenerative disease or disorder, organ tissue rejection, organ transplant rejection, or any combination thereof, etc.) from the subject or from the biological sample obtained from the subject.

### Method of Assessing the Prognosis of Esophageal Cancer

The present invention further relates, in part, to a method of assessing the prognosis of esophageal cancer in a subject in need thereof.

In one aspect, the present invention provides a method of assessing the prognosis of esophageal cancer in a subject, the method comprising obtaining a biological sample from the subject; measuring the abundance of at least one bacteria, at least one bacterial protein, at least one protein from the subject, or a combination thereof.

In some embodiments, the at least one bacterial protein is one or more selected from the group consisting of: a phage protein, a ribosomal protein, an MFS transporter, a protein linked to mitochondrial function, and an iron-sulfur cluster protein. Methods of measuring protein are discussed elsewhere herein.

In some embodiments, the at least one bacterial protein is one or more selected from the group consisting of: a phage protein, a ribosomal protein, an MFS transporter, a protein linked to mitochondrial function, and an iron-sulfur cluster protein.

In some embodiments, the protein linked to mitochondrial function is selected from the group consisting of pyruvate dehydrogenase, succinate dehydrogenase and aconitase. In some embodiments, the iron-sulfur cluster protein is selected from the group consisting of aconitase, succinate dehydrogenase iron-sulfur, and iron-sulfur cluster assembly SufB.

In some embodiments, a decrease in a protein linked to mitochondrial function, an iron-sulfur protein, or a combination thereof, relative to the comparator indicates the subject has a poor prognosis. In some embodiments, an increase in at least one bacterial protein selected from the group consisting of a phage protein, a ribosomal protein, an MFS transporter relative to the comparator indicates the subject has a poor prognosis.

In some embodiments, an increase in a protein linked to mitochondrial function, an iron-sulfur protein, or a combination thereof, relative to the comparator indicates the subject has a good prognosis. In some embodiments, a decrease in at least one bacterial protein selected from the group consisting of a phage protein, a ribosomal protein, an MFS transporter relative to the comparator indicates the subject has a good prognosis. In some embodiments, the at least one protein from the subject is one or more selected from the group consisting of SAT1, SAT2, FTL, MAP11C3B2, MAP11C3B, and VDAC2. Methods of measuring protein are discussed elsewhere herein.

In some embodiments, an increase in the at least one protein from the subject relative to the comparator indicates the subject has a poor prognosis. In some embodiments, a decrease in the at least one protein from the subject relative to the comparator indicates the subject has a good prognosis.

Information obtained from the methods of the invention described herein can be used alone, or in combination with other information (e.g., age, family history, disease status, disease history, vital signs, blood chemistry, PSA level, Gleason score, primary tumor staging, lymph node staging, metastasis staging, expression of other gene signatures relevant to outcomes of a disease or disorder, such as autoimmune disease or disorder, cancer, inflammatory disease or disorder, metabolic disease or disorder, neurodegenerative disease or disorder, organ tissue rejection, organ transplant rejection, or any combination thereof, etc.) from the subject or from the biological sample obtained from the subject.

### Method of Treatment

The present invention is, in part, related to the finding that bacteria, bacterial protein, protein from the subject, or a combination thereof are present or absent in esophageal cancer.

In some embodiments, the method of the invention further comprises administering a composition comprising a modulator of at least one bacteria, at least one bacterial protein, at least one protein from the subject, or a combination thereof to a subject in need. In some embodiments, the subject has esophageal cancer.

In some embodiments, the modulator increases the abundance of one or more bacteria from the genera selected from the group consisting of Cutibacterium, Sphigomonas, Fictibacillus, and Corynebacterium. In some embodiments, the modulator comprises one or more bacteria from the genera selected from the group consisting of Cutibacterium, Sphigomonas, Fictibacillus, and Corynebacterium. In some embodiments, the modulator decreases one or more bacteria selected from the group consisting of Bacillus, Gluconacetobacter, Peribacillus, Candidimonas, Burkholderia, Delfita, Halopseodomonas, Methylophilus, and Larkinella.

In some embodiments, the modulator increases the expression or activity of one or more bacterial protein selected from the group consisting of translation elongation factor EF-1 alpha, ferritin, NADHquinone oxidoreductase subunit H, a zincin-like metallopeptidase protein, DNA topoisomerase III, and a transposase. In some embodiments, the modulator decreases the expression or activity of one or more bacterial protein selected from the group consisting of a phage replicative protein, acyl-CoA dehydrogenase, LLM-class flavin dependent oxidoreductase, an ABC transporter component, a peptidase, an S49 peptidase, and a phosphatase. In some embodiments, the modulator decreases the expression or activity of one or more bacterial protein selected from the group consisting of a phage protein, a ribosomal protein, an MFS transporter. In some embodiments, the protein linked to mitochondrial function is selected from the group consisting of pyruvate dehydrogenase, succinate dehydrogenase and aconitase. In some embodiments, the iron-sulfur cluster protein is selected from the group consisting of aconitase, succinate dehydrogenase iron-sulfur, and iron-sulfur cluster assembly SufB.

In some embodiments, the modulator decreases the expression and/or activity of one or more host protein selected from the group consisting of: SAT1, SAT2, FTL, MAP11C3B2, MAP11C3B, and VDAC2.

In some embodiments, the modulator is one or more selected from the group consisting of a bacteria, chemical compound, a protein, a peptide, a peptidomemetic, an antibody, a ribozyme, a small molecule chemical compound, a nucleic acid, a vector, and an antisense nucleic acid molecule.

In some embodiments, the modulator is an inhibitor. In some embodiments, the inhibitor diminishes the amount of a target polypeptide, the amount of a target protein, the amount of target mRNA, the amount of a target enzymatic activity, or a combination thereof. In some embodiments, the target is one or more bacterial protein selected from the group consisting of a phage replicative protein, acyl-CoA dehydrogenase, LLM-class flavin dependent oxidoreductase, an ABC transporter component, a peptidase, an S49 peptidase, and a phosphatase. In some embodiments, the target is one or more bacterial protein selected from the group consisting of a phage protein, a ribosomal protein, an MFS transporter. In some embodiments, the protein linked to mitochondrial function is selected from the group consisting of pyruvate dehydrogenase, succinate dehydrogenase and aconitase. In some embodiments, the iron-sulfur cluster protein is selected from the group consisting of aconitase, succinate dehydrogenase iron-sulfur, and iron-sulfur cluster assembly SufB. In some embodiments, the target is one or more host protein selected from the group consisting of: SAT1, SAT2, FTL, MAP11C3B2, MAP11C3B, and VDAC2.

In some embodiments, the modulator is an activator. In some embodiments, the activator increases the amount of a target polypeptide, the amount of a target protein, the amount of target mRNA, the amount of a target enzymatic activity, or a combination thereof. one or more bacterial protein selected from the group consisting of translation elongation factor EF-1 alpha, ferritin, NADHquinone oxidoreductase subunit H, a zincin-like metallopeptidase protein, DNA topoisomerase III, and a transposase.

It will be understood by one skilled in the art, based upon the disclosure provided herein, that a decrease or increase in the level of the target encompasses the decrease or increase in target expression, including transcription, translation, or both, and also encompasses promoting or inhibiting the degradation of the target, including at the RNA level (e.g., RNAi, shRNA, etc.) and at the protein level (e.g., Ubiquitination, etc.) The skilled artisan will also appreciate, once armed with the teachings of the present invention, that a decrease or increase in the level of the target includes a decrease or increase in a target activity (e.g., enzymatic activity, substrate binding activity, receptor binding activity, etc.). Thus, decreasing or increasing the level or activity of the target includes, but is not limited to, decreasing or increasing transcription, translation, or both, of a nucleic acid encoding the target; and it also includes decreasing or increasing any activity of a target polypeptide, or peptide fragment thereof, as well.

The inhibitor or activator of the invention that decrease or increase the level or activity (e.g., enzymatic activity, substrate binding activity, receptor binding activity, etc.) of the target, include, but should not be construed as being limited to, a chemical compound, a protein, a peptide, a peptidomimetic, an antibody, an antibody fragment, a monobody, an antibody mimetic, a ribozyme, a small molecule chemical compound, an short hairpin RNA, RNAi, an antisense nucleic acid molecule (e.g., siRNA, miRNA, etc.), a nucleic acid encoding an antisense nucleic acid molecule, a nucleic acid sequence encoding a protein, or combinations thereof. In some embodiments, the inhibitor or activator is an allosteric inhibitor or activator. One of skill in the art would readily appreciate, based on the disclosure provided herein, that as inhibitor or activator of the target encompasses any chemical compound that decreases or increases the level or activity of the target. Additionally, an inhibitor or activator of the target encompasses a chemically modified compound, and derivatives, as is well known to one of skill in the chemical arts.

Further, one of skill in the art, when equipped with this disclosure and the methods exemplified herein, would appreciate that an inhibitor or activator of the target includes such inhibitors or activators as discovered in the future, as can be identified by well-known criteria in the art of pharmacology, such as the physiological results of inhibition or activation of the target as described in detail herein and/or as known in the art. Therefore, the present invention is not limited in any way to any particular inhibitor or activator as exemplified or disclosed herein; rather, the invention encompasses those inhibitor or activator that would be understood by the routineer to be useful as are known in the art and as are discovered in the future.

Further methods of identifying and producing inhibitor or activator of the target are well known to those of ordinary skill in the art, including, but not limited, obtaining an inhibitor or activator of the target from a naturally occurring source. Alternatively, an inhibitor or activator of the target can be synthesized chemically. Further, the person of skill in the art would appreciate, based upon the teachings provided herein, that an inhibitor or activator of the target can be obtained from a recombinant organism. Compositions and methods for chemically synthesizing inhibitors or activators of the target and for obtaining them from natural sources are well known in the art and are described in the art.

One of skill in the art will appreciate that an inhibitor or activator of the target can be administered as a chemical compound, a protein, a peptide, a peptidomimetic, an antibody, an antibody fragment, an antibody mimetic, a ribozyme, a small molecule chemical compound, a short hairpin RNA, RNAi, an antisense nucleic acid molecule (e.g., siRNA, miRNA, etc.), a nucleic acid encoding an antisense nucleic acid molecule, a nucleic acid sequence encoding a protein, or a combination thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor or activator of the target.

One of skill in the art will realize that diminishing or increasing the amount or activity of a molecule that itself increases or decreases the level or activity of the target can serve in the compositions and methods of the present invention to decrease or increase the level or activity of the target.

Antisense oligonucleotides are DNA or RNA molecules that are complementary to some portion of an RNA molecule. When present in a cell, antisense oligonucleotides hybridize to an existing RNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the amount of the target, or to diminish the amount of a molecule that causes an increase in the amount or activity of the target, thereby decreasing the amount or activity of the target.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited or activated by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing the target, or of a gene expressing a protein that increases the level or activity of the target, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28:4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

Alternatively, inhibition or activation of a gene expressing the target, or of a gene expressing a protein that decreases or increases the level or activity of the target, can be accomplished through the use of a short hairpin RNA or antisense RNA, including siRNA, miRNA, and RNAi. Given the nucleotide sequence of the molecule, one of ordinary skill in the art could synthesize a short hairpin RNA or antisense RNA without undue experimentation, provided with the disclosure and references incorporated herein.

In one embodiment, the invention provides a method to treat cancer metastasis. In some embodiments, the method comprises diagnosing the subject with cancer comprising the methods described herein, and treating the subject with a therapy for cancer such as surgery, chemotherapy, chemotherapeutic agent, radiation therapy, or hormonal therapy or a combination thereof. In some embodiments, the method comprises treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Microbial Gene Expression Analysis of Healthy and Cancerous Esophagus Uncovers Bacterial Biomarkers of Clinical Outcomes

Several lines of emerging evidence point to a substantial role of tumor and resident microbes in cancer development and progression (Sepich-Poore et al., Science. (2021) 271: eabc4552; Wong-Rolle et al., Protein Cell. (2021) 12:426-35; Culin et al., Cancer Cell. (2021) 39:1317-41). Bulk tumor RNA sequencing can be utilized to study both intratumor and tumor-microenvironment microbial expression. However, existing short-read RNA sequencing datasets, which represent the largest source of cancer sequence information, are ill-suited for researching microbiomes. In particular, short nucleotide reads are very challenging to map accurately to individual microbial species or specific proteins. The naïve alternative to direct read mapping is an exhaustive assembly of sequencing reads to produce longer putative contigs, but this is computationally infeasible for all but the smallest sequencing datasets. Further, knowledge of a cancer microbiome has very limited diagnostic or prognostic value without comparison to a suitable non-cancerous control. While paired comparisons between cancer and nearby non-cancerous tissue are the most straightforward, microbiome disruptions that precede cancer may occur in nearby non-cancerous tissue as well. For example, canonical oncogenic viruses generally lead to cancer only after a persistent, often decades-long infection of the tissue of origin (Moore and Chang, Nat Rev Cancer. (2010) 10:878-89; Tornesello et al., Cancers. (2018) 10:213; Guven-Maiorov et al., Front Oncol. (2019) 9:1236), which is likely to be widespread relative to the cancer cell of origin.

A new method was developed to overcome many of these challenges in the characterization of bacterial populations from RNAseq. This method was applied to compare bacterial species and proteins in esophageal carcinoma (ESCA) and the healthy esophagus. To overcome the limitations of both direct mapping and naïve assembly, the approach first employs a deep learning model to identify RNAseq reads with likely bacterial or viral origin. These reads are then used as seeds in a targeted seed and extend assembly pipeline to produce longer candidate microbial contigs. These contigs were then mapped to curated databases of bacterial and viral nucleotide sequences, as well as bacterial protein families. To understand patterns in the ESCA microbiome at the population level, comparable RNAseq samples from hundreds of healthy esophagi as a robust noncancerous control were used.

Substantial differences were found in the complements of bacterial taxa and bacterial protein products between ESCA samples and the healthy population. Most genera with nontrivial prevalence in one population were present at significantly different rates, with the majority more abundant in healthy esophagi. Yet, surprisingly, genera whose presence is significantly correlated with outcome among the ESCA patients were not found. In contrast, most bacterial protein families with a significant difference in prevalence were more commonly detected in cancers, although this might be attributable to variations in sequencing depth enabling the detection of proteins with a lower level of expression in the ESCA samples.

Surprisingly, about half of the top bacterial proteins identified as overexpressed in cancer are derived from phages. Bacteriophages may alter microbiomes by disproportionally infecting certain bacterial species and by facilitating gene transfer (Kato et al., Cancers. (2022) 14:425). Therefore, certain combinations of phages could favor cancer-associated bacteria. Several bacterial protein families whose presence is also associated with outcomes in ESCA patients were found. Further, bacterial expression of iron-sulfur proteins in ESCA was associate with altered expression of host genes. The affected human genes included several in the ferroptosis pathway, an alternate cell death pathway, that was independently associated with poor outcomes. One possible mechanism to link ferroptosis dysregulation with poor patient outcomes is through iron excess and ferroptosis resistance, supported by upregulation of FTL, which stores iron and is upregulated in ferroptosis resistant cells (Xie et al., Cell Death Differ. (2016) 23:369-79). Excess iron beyond iron storage capacity allows for redox-active iron and oxidative stress (Galaris et al., Biochim Biophys Acta Mol Cell Res. (2019) 1866:118535). Indeed, several microbial genes associated with ESCA outcomes confer mitochondrial functions and were linked with host oxidative phosphorylation. Importantly, mitochondrial oxidative phosphorylation is increasingly recognized as a key mechanism for metabolic reprogramming in cancer (Faubert et al., Science. (2020) 368: eaaw5473; Vasan et al., Cell Metab. (2020) 32:341-52).

All code and scripts associated with this work are publicly and freely available through GitHub: github.com/AuslanderLab/virnatrap-bacteria.

The methods are described herein.

### Model Training

To classify reads, a model was trained to predict the origin of a 76-base pair sequence from among human, viral, and bacterial. To simulate RNAseq reads from each class, segmentation into 76-base sequences was performed to (1) the human hg19 reference transcriptome, obtained from NCBI (Sayers et al., Nucleic Acids Res. (2021) 49: D10-7), (2) a database of transcripts from diverse viruses of placental mammals, obtained from the Virus Variation Resource (Hatcher et al., Nucleic Acids Res. (2017) 45: D482-90), and (3) a database of bacterial genomes containing one representative per genus, curated previously (Auslander et al., Nucleic Acids Res. (2020) 48: e121). To generate balanced data, sequences were segmented with stride two for viral sequences, stride 26 for human sequences, and stride 130 for bacterial sequences. Sequences were randomly divided into training, validation, and testing sets; this split was done before segmenting. Segments containing N's were excluded. This yielded a training set of size 21,005,972 (7,000,098 human, 6,996,574 viral, 7,009,300 bacterial), a validation set of size 4,503,578 (1500036, 1498065, 1505477), and a testing set of size 5,628,298 (1873416, 1863322, 1891560). To predict the likely origin of reads, a small convolutional neural network was trained, with two convolutional layers and one fully-connected layer. Hyperparameters were tuned and the best performing model by one-versus all area under the precision-recall curve (AUPRC) on the validation set was selected. All models were trained using TensorFlow 2.8 (Abadi et al., (2016) arxiv 1603.04467).

### Sequence Assembly and Identification

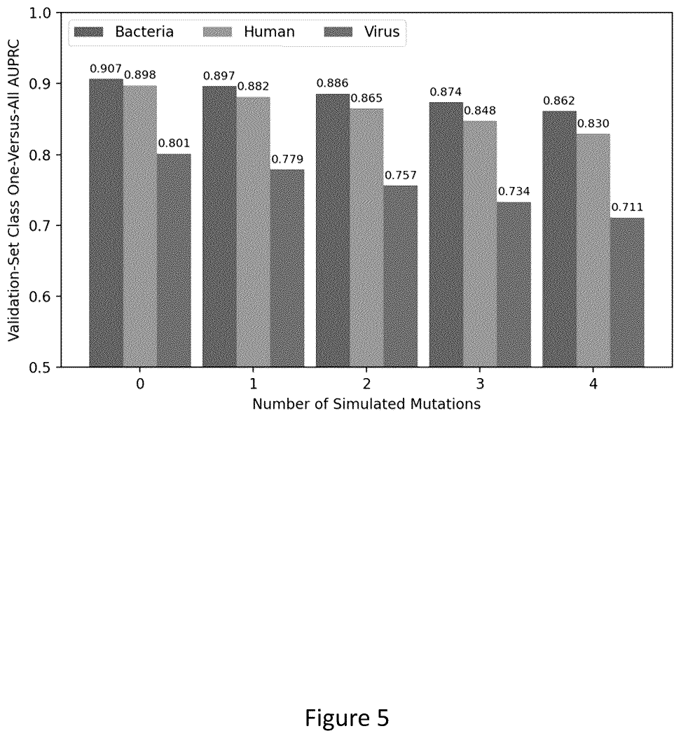

75-base RNAseq reads were obtained from 170 esophageal carcinomas through TCGA (Cancer Genome Atlas Research Network et al., Nature. (2017) 541:169-75) and 76-base reads from 1565 healthy esophageal samples from 742 unique individuals through GTEx (Lonsdale et al., Nat Genet. (2013) 45:580-5). These projects used similar RNAseq protocols (The Cancer Genome Atlas Research Network, Nature. (2014) 513:202-9); briefly, total RNA was isolated, polyadenylated RNAs were enriched (eukaryotic mRNAs are 3′ polyadenylated), cDNA was synthesized from the RNA, amplified, and purified, and reads were sequenced using the Illumina HiSeq 2000. Reads that map to the human genome were removed using the hg19 reference. Model scores assigned to each read were obtained, denoting the relative likelihoods of human, viral or bacterial origins. For prediction and assembly all reads with more than one N (0.17% of unmapped TCGA reads; 0.57% of unmapped GTEx reads) were excluded. Overall, 2,656,993,271 TCGA reads and 631,388,801 GTEx reads were considered. For reads with one N (0.22% of unmapped TCGA reads; 3.74% of unmapped GTEx reads), the N was replaced with a random nucleotide for prediction only. TCGA reads, again for prediction only, were padded with a random 3′ nucleotide to match the 76-base length expected by the model. On the validation data, replacing only one or two nucleotides with a random replacement had only a small impact on model performance (FIG. 5).

Once human, bacterial, and viral model scores were assigned to each read, those predictions were used to guide assembly of the reads into larger sequences. Every read with a bacterial or viral score of at least 0.46 was considered to be a “seed” read (FIG. 5). To prioritize sequences that were (1) likely to be microbial and (2) likely to be bacterial, the seed reads were sorted to first take likely bacterial seeds in descending bacterial score order and then likely-viral seeds in descending viral score order. For each seed, a longer sequence assembly was attempted by greedily extending the seed in each direction using a modification of the assembly tool developed previously (Elbasir et al., Nat Commun. (2023) 14:1-12). For assembly, an N was considered to match any nucleotide and, when such a match happened during extension, the non-N nucleotide was kept.

### Mapping Assembled Microbial Sequences to Bacterial Taxa

The resulting putative microbial species present in each sample were identified by comparing them to several curated databases of microbial nucleotide sequences using blastn (Altschul et al., J Mol Biol. (1990) 215; 403-10). For bacterial sequences, the set of NCBI representative bacterial genomes were used (approximately one per bacterial species). Two databases of viral RNA sequences were used, one for ‘reference’ human viruses and the other for ‘novel’ or non-human viruses, curated previously (Elbasir et al., Nat Commun. (2023) 14:1-12). Hits were filtered with e-value below 0.01 and assigned the sequence and species from the top BLAST hit to each sequence. For characterizing the abundance of organisms in cancer, all species at the genus level were pooled to reduce the number of hypotheses and to reflect the possible inaccuracy of identifying short sequences at the species level.

### Over and Under Representation of Microbial Genera

The prevalence of bacterial genera in ESCA and healthy esophagus were compared. The prevalence of each genus in each sample was computed, pooling all species in each genus. Occurrences in multiple esophagus samples from the same patient were also pooled. Overall, at least one bacterial transcript in all 161 ESCA cases and in healthy esophagus samples from 742 distinct patients were identified. Those genera that occurred in at least 10% of ESCA or 10% of healthy samples were selected as genera of interest. To quantify bacterial over- or underabundance in cancer, a one-tailed binomial test, using the binom_test method from scipy 1.10 were performed (Virtanen et al., Nat Methods. (2020) 17:261-72). For each genus, the hypothesized probability was set to be the fraction of healthy samples in which the genus was detected, except that minimum and maximum probabilities of 0.0001 and 0.9999 were used, as using exactly 0 or 1 would always produce a p-value of 0. The number of successes were then specified as the number of ESCA samples in which the genus was detected, the number of trials as 161, and the hypothesis as “less” or “greater” depending on whether the ESCA abundance was lower or higher than the healthy abundance. P-values were corrected using Benjamini-Hochberg FDR correction (Benjamini et al., J R Stat Soc. (1995) 57:289-300).

### Confounder Corrected Analysis for Over and Under Representation of Microbial Genera and Proteins

In addition to the analysis described above, a similar analysis was performed when correcting for possible confounders, such as clinical and background differences between TCGA and GTEx cohorts. Therefore, 715 individuals from GTEx and 122 cases from TCGA were used with complete background information to perform the analysis (that is, with race, age, sex, weight, and smoking information). Additionally, the sequencing depth of each sample was included as a cofounder in the corrected analysis, using the average sequencing depth for individuals with multiple samples. Chi-squared test was performed, which is appropriate for this large dataset with hundreds of samples. To adjust for confounders, a boosted logistic regression model was first fitted with confounders as covariates to estimate the probabilities of being in the TCGA vs GTEx cohorts. The resulting AUC (area under the curve) was 1.00, indicating substantial differences between the cohorts based on these confounders. Then, weighted Chi-squared tests were performed to evaluate bacterial under and over representation, where the weights are the inverse of estimated probabilities of being in the TCGA vs GTEx groups. In the weighted data, the covariates are balanced between the TCGA and GTEx groups. Therefore, using the weighted chi-squared test allowed for mitigating confounders in the evaluation of bacterial under and over representation in TCGA vs GTEx groups. For this analysis, all bacterial genera with any abundance were considered. FDR correction (Benjamini et al., JR Stat Soc (1995) 57:289-300) was then used to correct for multiple hypotheses. An identical approach was used to perform a corrected analysis for the over- or underprevalence of microbial protein families, which were identified as described below.

### Phylogenetic Analysis

A tree of selected bacterial genera was created by obtaining 16S rRNA gene sequences, one per genus, from GenBank, choosing a RefSeq sequence if available. These sequences were then aligned using MUSCLE version 5.1 (Edgar, Nucleic Acids Res. (2004) 32:1792-7; Edgar, Biorxiv. (2020) 449169). with default parameters, and constructed a tree using FastTree version 2.1.11 (Price et al., PLOS ONE. (2010) 5: e9490) with default parameters. The tree was visualized using iTOL (Letunic and Bork, Nucleic Acids Res. (2021) W293-6).

### Survival Analyses

To evaluate the association between bacterial species and ESCA survival the presence of each individual species was correlated (for which at least 5 positive and 5 negative ESCA samples were identified; excluding samples with no clinical data) with overall and disease stable survival using the logrank test through Python lifeline package (Davudson-Pilon, J Open Source Softw. (2019) 4:1317). TCGA clinical information was obtained through the TCGA Clinical Data Resource (Liu et al., Cell. (2018) 173:400-416.e11). This (meta) dataset includes, among other measures, both overall survival, which measures time to the death of a patient, and disease-free survival, which measures the time until cancer recurs after primary therapy. Log-rank p-values estimating association between expression of different bacterial genera and overall and disease-free survival were FDR-corrected for multiple comparisons, where no significant association was found. To evaluate the association between microbial proteins and survival, overall and disease-free survival for patients positive and negative for the expression of each microbial protein was similarly compared (for which at least 5 positive and 5 negative ESCA samples are identified). Several microbial proteins were identified that were significantly associated with survival after FDR correction for multiple comparisons.

### Mapping Assembled Contigs to Microbial Genes

The assembled contigs to microbial genes were mapped through RefSeq nonredundant microbial sequence database, downloaded from NCBI through the non-redundant proteins annotated on representative genomes. Contigs were mapped using blastx, with e-value below 1e-5. Presence or absence of each microbial gene in each sample considered were used for further analysis. For these analyses, 155 of the 170 ESCA samples with available clinical information were considered. Where healthy esophagus contigs were used, all 1565 samples were considered.

### Host Gene Expression Analyses

To evaluate host correlates of microbial iron-related (Fe) genes, human gene expression data of TCGA ESCA samples were analyzed. RNAseq RSEM values for ESCA samples were downloaded from cBioportal (Cerami et al., Cancer Discovery. (2012) 2:401-4; Gao et al, Sci Signal. (2013) 6:11). The expression of all human genes was compared between samples positive vs those negative for microbial Fe proteins that were found significantly associated with poor outcomes (accessions WP_006680945.1, WP_002532908.1 and WP_131625607.1) using a rank-sum test. None of the genes were significantly associated with microbial Fe-gene presence after FDR correction for multiple comparisons. To evaluate the processes that were upregulated in these samples, human genes assigned with unadjusted p-value <0.05, and where the median z-score for Fe-positive samples was above 0.2, and that for Fe-negative samples was below 0 were extracted. KEGG enrichment (Kanehisa et al., Nucleic Acids Res. (2016) 44: D457-62). was used to identify host (human) pathways enriched with genes upregulated in microbial Fe-positive ESCA samples.

### Genome Scale Metabolic Modeling

To compare oxygen consumption and ATP production rates between ESCA samples that are positive or negative for microbial genes associated with poor survival, genome scale metabolic modeling (GSMM) was used. The GIMME algorithm (Becker et al., PLOS Comput Biol. (2008) 4: e1000082) was used to constrain each metabolic model by the gene expression values in each ESCA sample, and applied Flux Balance Analysis (FBA) (Price et al., Nat Rev Microbiol. (2004) 2:886-97) to generate a predicted metabolic flux for each sample. The Recon1 human metabolic model (Duarte et al., Proc Natl Acad Sci USA. (2007) 104:1777-82) and the COBRA Toolbox v.3.0 implementation of GSMM functions (Heirendt et al., Nat Protoc. (2019) 14:639-702) was used.

### Model Training: Detailed Model Architecture and Training Procedures

A convolutional neural network was trained, consisting of an embedding layer, two 1D convolutional layers with 64 filters each of width 64 and padding with zeros, a max-pooling layer with width 9 (and stride 1), one fully connected layer with 64 units, all with ReLU activation, and an output layer with SoftMax activation. The learning rate was set to 0.0001, and L2 normalization with weight 0.01 was used.

During training, hyper-parameter tuning was performed over the number of convolutional layers and units, the number of fully connected layers and units, the width of the convolutions, and the width of the max pool. Limited tuning of the learning rate and dropout was also performed. Models were compared based on validation-set one-versus-all area under the precision-recall curve (AUPRC).

All models were trained using TensorFlow 2.8 for 100 epochs using the Adam optimizer, treating the number of epochs as a hyperparameter. Most hyperparameter tuning was performed by training models on a randomly-selected quarter of the training dataset, which we observed to produce only a marginal decrease in training-set performance. Additionally, during hyperparameter tuning, approximately 4,000 sequences containing ambiguous nucleotides other than N, all encoded as A, were erroneously included in the training data. The final model was retrained on the full training set and with sequences containing ambiguous nucleotides excluded.

Sequence Assembly and Identification: Assembling Sequences from Seed Reads

For each seed read, a longer sequence was assembled by greedily extending the seed in each direction using a modification of the assembly tool developed for viRNAtrap. Specifically, the terminal 24-mer of the current sequence in all other reads was searcged, and then, if at least one match was found, extended with the matching read that gave the largest extension.

All matching reads were considered consumed and ineligible for inclusion into another sequence. Additionally, any reads that were found to be wholly contained in each contig were excluded from any future contig. Where applicable, an N was considered to match against any nucleotide, and when an N was aligned against another nucleotide in the assembly on a contig the non-N was always kept.

Survival Analyses: Association of Bacterial Species and Proteins with Survival

All survival analyses were performed by comparing the presence vs. absence of each bacteria species or protein. Significance was evaluated using the log-rank test, through Python lifelines.statistics.StatisticalResult v0.27.4. P-values were FDR-corrected for multiple comparisons. Survival curves were fitted and visualized using Kaplan Meier curves, through Pythom lifelines.fitters.kaplan_meier_fitter.KaplanMeierFitter.

Non-Associations of Host Genes with Patient Survival

The ferroptosis host genes that are upregulated in bacterial Fe-positive samples include SAT1 as well as SAT2 which have been linked to improved outcomes in several adenocarcinomas. A similar survival analysis was applied, using the expression of SAT1, SAT2 and the z-score combining SAT1 and SAT2, all of which were not significantly associated with survival. SAT1 and SAT2 are not individually associated with better survival in ESCA, and that their combined expression with the other ferroptosis host genes identified is associated with poor survival.

### Identifying Common Sequencing Contaminants

The list of collected contaminants, including vector contaminants and different sequence artifacts that were identified previously for viRNAtrap were used. These were used to filter out assembled contigs from being mapped to microbial species or genes. Any accessions associated with contaminants were entirely removed from the search.

The results are described herein.

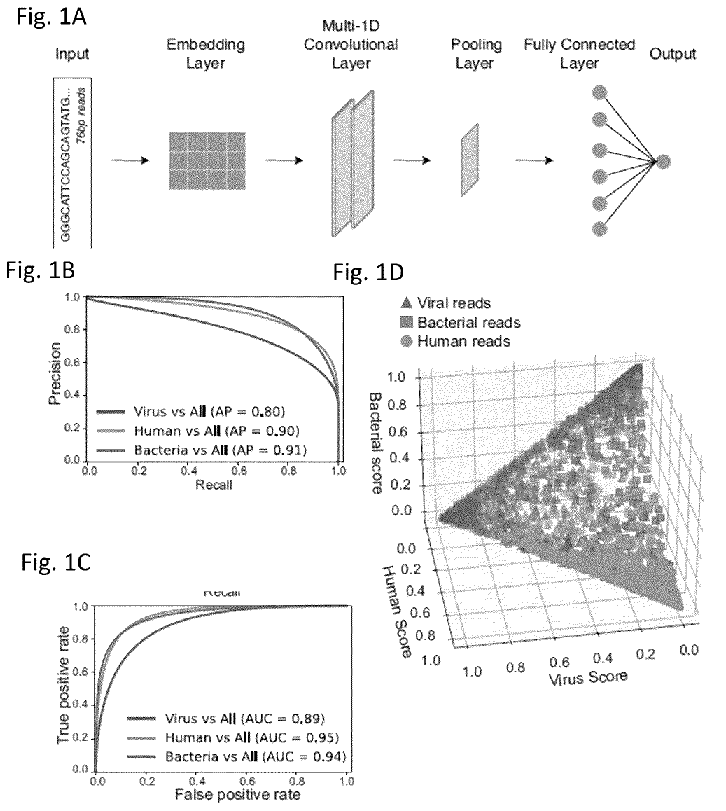

To allow alignment free prediction of viruses and bacteria from short-read RNAseq data, a convolutional neural network was trained to classify 76-base nucleotide sequence as having human, viral, or bacterial origins (FIG. 1A). To simulate RNAseq reads for training, segmented sequences from the human transcriptome, viral transcriptomes, and bacterial genomes were used. Dozens of convolutional neural networks were trained with varying hyperparameters and selected the model with the best performance on a held-out validation set. The final model was then evaluated on a separate test set of held-out human, viral, and bacterial sequences (FIG. 1B-FIG. 1D). It demonstrated one-versus-all Area Under the Precision-Recall Curve (AUPRC) of 0.89 for human sequences, 0.91 for bacterial sequences, and 0.80 for viral sequences. The best possible AUPRC is 1.0, corresponding to a perfect classifier, while the AUPRC of a random classifier is equal to the fraction of positive examples, which is about 0.33 in the balanced three-class case. The model further demonstrated Area Under the Receiver-Operating Curve (AUROC) of 0.95 for human sequences, 0.94 for bacterial sequences, and 0.89 for viral sequences. The best possible AUROC is 1.0, corresponding to a perfect classifier, while the AUROC of a random classifier is 0.5.

The model serves as the first step of the pipeline to identify bacterial and viral pathogens from RNAseq data. Starting with unmapped RNAseq reads, predictions from the model are used to guide assembly into longer putative-pathogenic contigs. Then, these contigs are aligned to broad databases of viral and bacterial genomes to detect those that are expressed in each sample. This pipeline was applied to study the prevalence of viruses and bacteria in esophageal cancer, using RNAseq data from cancer patients (obtained via TCGA) as well as from a larger population of healthy control esophagi (obtained via GTEx). Using the labeled contigs produced by the pipeline, bacterial genera that are under or overrepresented in cancer were first searched.

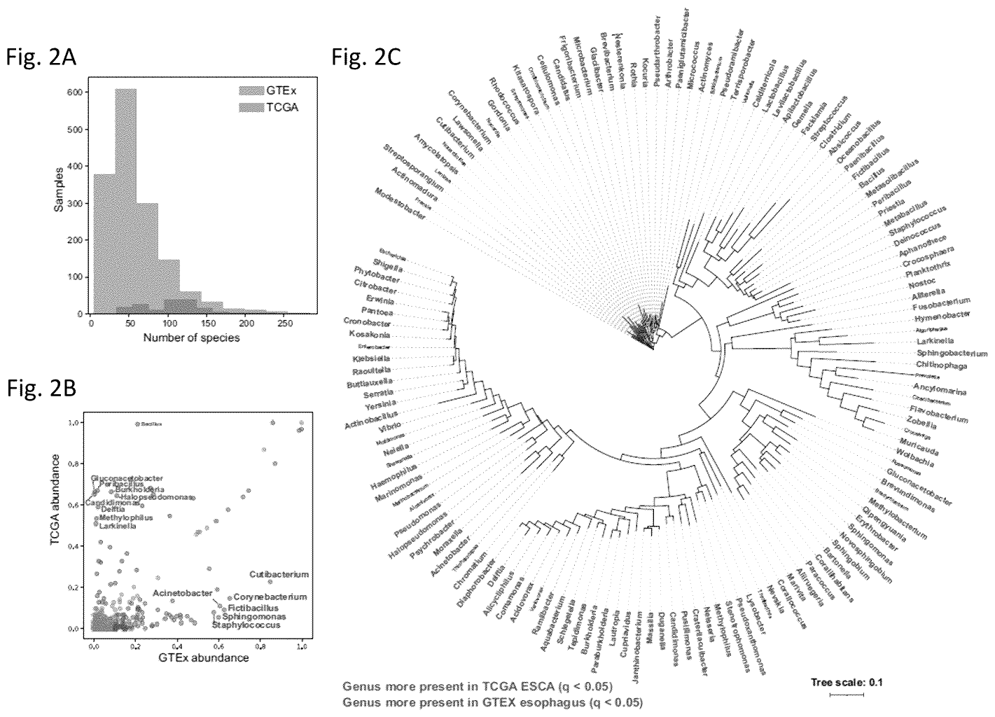

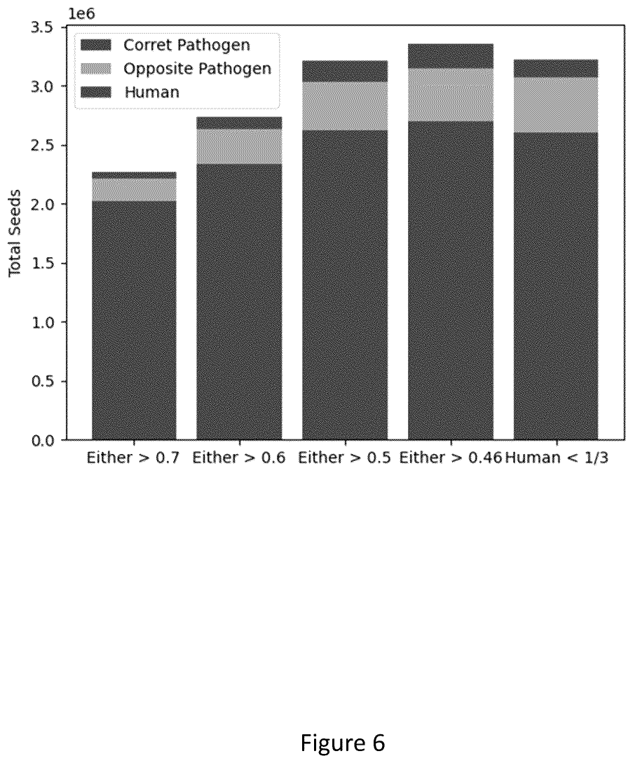

Overall, sequences from 161 ESCA cases and 742 healthy esophagi were attributed to 6,961 unique bacterial species (FIG. 2A). Considering 145 genera that are sufficiently represented in the data (FIG. 2B), and applying a permissive threshold for presence of one contig, 32 genera that were significantly over-prevalent in cancer and 90 that were significantly under-prevalent in cancer were found (pFDR <0.05; FIGS. 2B, FIG. 2C, and FIG. 6). This analysis was additionally performed controlling for possible confounders and differences between the cohorts, including the sequencing depth of each sample. The cancer under-abundant bacterial genera are particularly notable, as the read depth and number of species found were both lower for the GTEx samples compared to TCGA samples, despite lower sequencing depth (FIG. 2B). Because of the sample size, even small absolute differences in abundances can be significant (FIG. 2B).

The genera with the largest absolute differences best distinguish the cancer and healthy conditions. Among the 90 underabundant genera, four occur in at least 50 percentage points fewer ESCA samples than healthy: Cutibacterium, Sphigomonas, Fictibacillus, and Corynebacterium (FIG. 2B and FIG. 2C). The family Sphingomonadaceae, which includes Sphigomonas, was previously suggested to be protective against breast cancer (Lawani-Luwaji et al., Bull Nat Res Cent. (2020) 44:191). The highlighted bacterium in that study was a member of the genus Sphingobium, which was found in 18.3% of healthy esophagi but only a single ESCA sample (FIG. 2B and FIG. 2C). Additionally, Corynebacterium parvum was first reported to promote an immune response and survival in cancer more than 40 years ago (Scott, Semin Oncol. (1974) 1:367-78; Knapp and Berkowitz, Am J Obstet Gynecol. (1997) 128:782-6).

Among the 32 overabundant genera, nine occur in at least 50 percentage points more ESCA samples than healthy: Bacillus, Gluconacetobacter, Peribacillus, Candidimonas, Burkholderia, Delfita, Halopseodomonas, Methylophilus, and Larkinella (FIG. 2B and FIG. 2C). Most of these genera occur in a very small fraction of healthy esophagi and a bit more than half of ESCA samples. However, most striking is the common genus Bacillus, which was detected in all but one ESCA sample for which any bacterial sequences were detected, but only 21% of healthy esophagi. Aside from the closely-related Bacillus and Peribacillus, as well as the unique Larkinella, the other genera six genera represent Alpha-, Beta-, or Gamma-Proteobacteria. Interestingly, increased Proteobacteria abundance was previously reported in pancreatic and breast cancers (Pushalkar et al., Cancer Sicov. (2018) 8:403-16; Fernandez et al., Int J Environ Res Public Health. (2018) 15:1747), and was previously reported in nine cancer types from TCGA (Rodriguez et al., Comput Struct Biotechnol J. (2020) 18:631-41). At the genus and clade level, these increases of common taxa may represent an overall increase in bacterial load in ESCA, or may be linked to tissue and microenvironment differences between the cohorts. On the other hand, members of the small genus Larkinella (class Cytophagales), which have been isolated from diverse environments, principally soil (Park et al., Arch Microbiol. (2022) 204:182; Zhou et al., Arch Microbiol (2020) 202:2517-23; Pelletier et al., Microbiol Resour Announc. (2020) 9: e00159-20; Xu et al., Int J Syst Evol Microbiol (2017) 67:5134-8; Anandham et al., Int J Syst Evol Microbiol (2011) 61:30-4), were identified by one study in bladder cancer, reporting an association between Larkinella and recurrence (Zeng et al., Front Cell Infect Microbiol (2020) 10:555508).

Interestingly, very low levels of Helicobacter were found (including H. pylori) in both GTEx samples (0.1%) and TCGA samples (0.6%). This supports the specificity of H. pylori as an oncogenic agent in stomach cancer only, and is consistent with previous studies and meta-analyses finding either no or a weak negative (protective) association between overall H. pylori infection and ESCA (Xie et al., World J Gastroenterol (2013) 19:6098-107; Gao et al., Gastroenterol Res Pract (2019) 1953497). In addition to bacteria, the presence of viral clades in with ESCA and healthy tissues were examined. Overall, matches to 691 unique viral strains in 61 ESCA samples and 503 healthy esophagi were found. The most common clade observed is herpesviruses, which were detected in 32 ESCA samples and 162 healthy esophagi. Strikingly, a Geobacillus bacteriophage was found in 192 healthy esophagi, where 181 were positive for type E2 and 98 were positive for type E3. Interestingly, however, Geobacillus bacteriophage was not detected a single ESCA sample. Surprisingly, Geobacillus was directly detected in only 17 esophagi, and detected both Geobacillus and a Geobacillus phage in only four esophagi. This could be explained by a possible different host of this bacteriophage, or enhanced expression of the bacteriophage compared to the bacterial host. Of additional note is a virus of the genus Vientovirus, DNA viruses that infect Entamoeba gingivalis (Keeler et al., Cell Host Microbe. (2023) 31:58-68.e5) and are found in the human mouth and respiratory tract (Abbas et al., Cell Host Microbe. (2019) 25:719-.e4), found in two ESCA samples.

Previous studies have suggested that the presence of specific bacteria in several tumors is correlated with survival (Mager et al., J Transl Med. (2005) 3:27; Riquelme et al., Cell. (2019) 178:795-806.e12; Yan et al., Gastroenterology. (2007) 132:562-75). bacterial species whose presence or absence in tumor RNAseq is correlated with the survival of ESCA patients was then searched. However, no significant associations were found.

Instead of the presence of a specific bacterial taxon, microbial processes executed by different bacteria may be associated with oncogenesis and therefore correlated with outcomes. This would be consistent with the large number of overabundant bacterial clades yet lack of species correlated with patient survival. Therefore, identifying specific microbial proteins that are expressed in ESCA and were identified and whether any such proteins correlate with outcomes was evaluated.

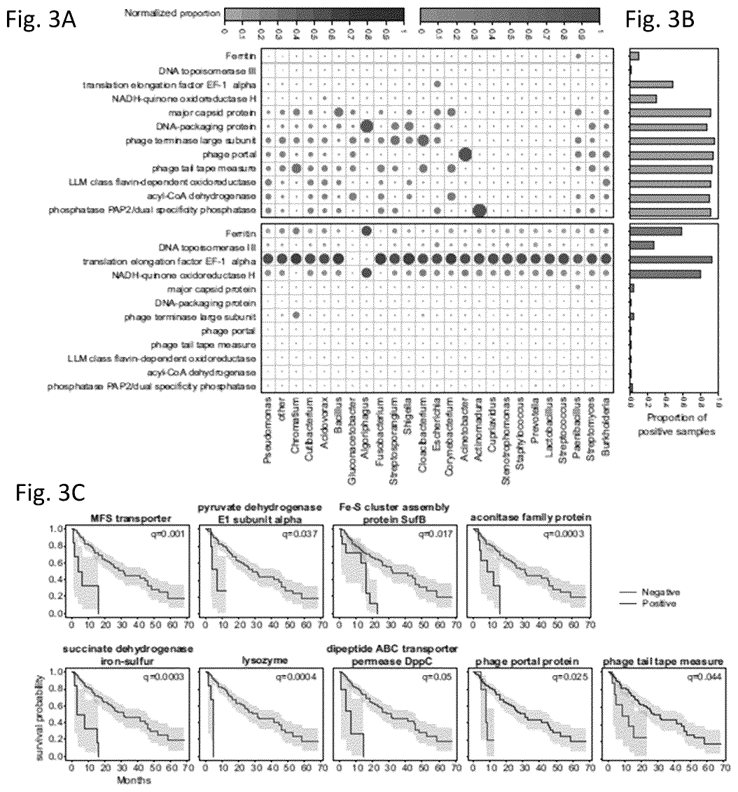

To that end, each microbial contig was mapped against a database of representative microbial proteins. Among all samples, transcripts of 16,261 bacterial proteins were identified, including transcription products of several notable gene families from diverse bacteria in both healthy and cancerous samples (FIG. 3A and FIG. 3B). As expected, the large majority (87.6%, N=14248) had little difference in prevalence between cancer and healthy (at most a 5-percentage-point difference in ESCA and healthy occurrences). However, some protein families did show considerable differences in prevalence. Only 21 were substantially more present in healthy esophagus (healthy frequency-ESCA frequency >25%). The top five include translation elongation factor EF-1 alpha, ferritin, NADHquinone oxidoreductase subunit H, and two unnamed protein products comprising nucleotide-binding domains. The healthy-abundant proteins also include a zincin-like metallopeptidase protein and DNA topoisomerase III, which are present in only 1.3% and 0.6% of ESCA samples, respectively, and several transposases. In contrast, 697 proteins were comparably overrepresented in the cancer samples (ESCA frequency—healthy frequency >25%). This asymmetry may be explained in part by the greater sequencing depth of ESCA samples—the average protein is present in 2.7% more ESCA samples than healthy esophagi. Most strikingly, phage replicative proteins are consistently more abundant in cancers (FIG. 3A and FIG. 3B), and the top over-present proteins in ESCA (occurring in 80 percentage points more ESCA samples, N=66) include at least 37 phage protein families. While many of these hits may be redundant, at least 7 phage components are represented in the top proteins. Other top cancer-abundant proteins include an acyl-CoA dehydrogenase, an LLM-class flavin dependent oxidoreductase, ABC transporter components, multiple peptidases including the S49 family, and multiple phosphatases (FIG. 3A and FIG. 3B). It was additionally found that, overall, more than 2000 protein families are significantly (q<0.05) differentially present after controlling for possible confounders and differences between the cohorts, including the sequencing depth of each sample.

Among the bacterial gene families found expressed in cancer samples, several are significantly associated with overall and disease particular, there are 34 families whose presence in the sample is significantly negatively associated with survival, although several were phage, ribosomal, or unlabeled proteins. Among the remainder, MFS transporters, of which hits to three representatives among the 34 families were found, comprise a diverse and ubiquitous class of multi-substrate membrane transport proteins (Madej et al., Proc Natl Acad Sci USA. (2013) 110:5870-4; Lewinson et al., Mol Microbiol. (2006) 61:277-84). While MFS transporters have a clinically-important role in antibiotic resistance (Lowrence et al., Crit Rev Microbiol. (2019) 45:1-20; Lewinson et al., Mol Microbiol. (2006) 61:277-84), their possible role in human cancers has not been elucidated. Specifically, removal of chemotherapy agents in drug-resistant cancers is generally performed by ABC transporters rather than human MFS homologs (Lowrence et al., Crit Rev Microbiol. (2019) 45:1-20). Lysozyme is a small antibacterial protein that principally targets bacterial cell walls, especially those of Grampositive bacteria (Ragland and Criss, Plos Pathog. (2017) 13: e1006512; Ferraboschi et al., Antibiotics. (2021) 10:1534). While it is primarily known as a multifunctional component of animal immunity (Ragland and Criss, Plos Pathog. (2017) 13: e1006512), lysozyme is produced by many organisms, including bacteria (Ferraboschi et al., Antibiotics. (2021) 10:1534), for microbial defense and competition.

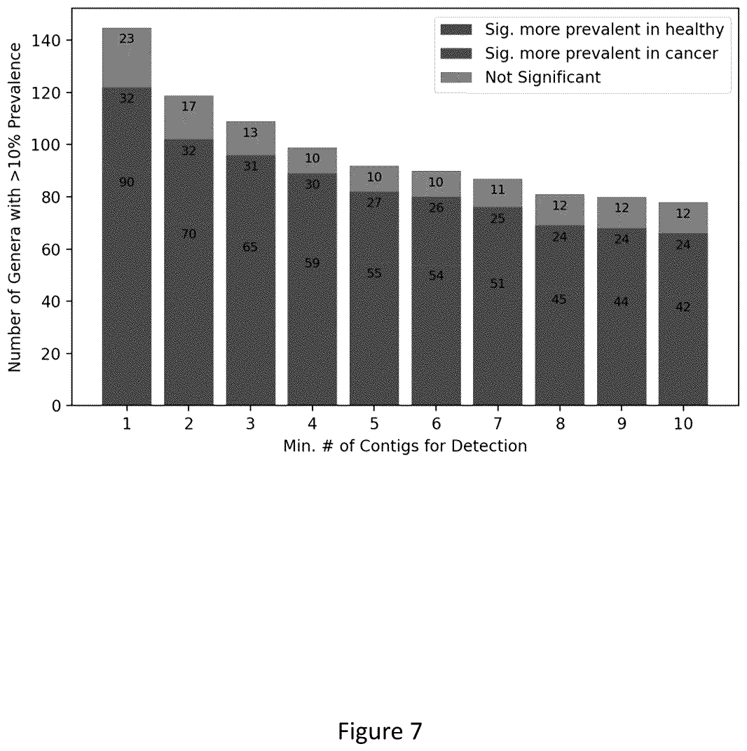

Among the microbial proteins that are significantly associated with survival, several are linked with mitochondrial functions, such as pyruvate dehydrogenase, succinate dehydrogenase and aconitase. This implies a possible metabolic shift in cancers expressing these microbial proteins, linked with enhanced complex II respiration and oxidative stress. Indeed, examining host gene expression, oxidative phosphorylation gene expression is elevated in samples positive for these microbial proteins (FIG. 7A). Furthermore, using genome scale metabolic modeling shows that oxygen consumption rates and ATP production are elevated in ESCA samples expressing these microbial proteins, supporting the notion that mitochondrial shift may be underlying the link between these proteins and poor patients' outcomes (FIG. 7B and FIG. 7C). Three protein families that are significantly associated with poor survival are microbial iron-sulfur cluster proteins: aconitase, succinate dehydrogenase iron-sulfur, and iron-sulfur cluster assembly SufB. Indeed, iron is required for bacterial proliferation (Crioss et al., Sci Rep. (2015) 5:16670; Nairz and Weiss, Mol Aspets Med. (2020) 75:100864). Therefore, whether the presence of these genes was correlated with changes in the human tumor transcriptome was investigated.

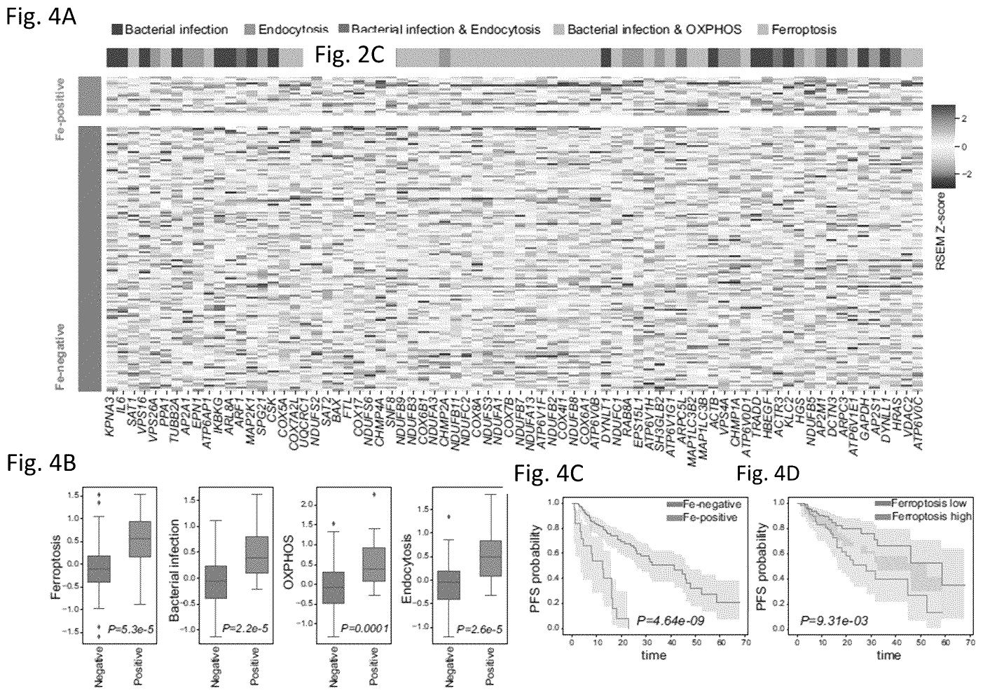

A large number of upregulated host genes in ESCA samples expressing microbial iron proteins were identified, across four key upregulated pathways: bacterial infection response, endocytosis, oxidative phosphorylation, and ferroptosis (FIG. 4A and FIG. 4B; Table 1). Ferroptosis, in particular, is a recently-characterized cell death pathway, with relevance to cancer progression (Lei et al., Nat Rev Cancer. (2022) 22:381-96). As observed with the individual gene families, presence of bacterial Fe-genes overall is negatively associated with survival (FIGS. 3C and 4C). Further, high expression of distinct host ferroptosis genes is itself associated with worse survival, in contrast to the three other pathways (FIG. 4D). These genes include SAT1, SAT2, FTL, MAP11C3B2, MAP11C3B, and VDAC2. Increased SAT1 expression, including by the p53 tumor suppressor, promotes the ferroptosis cell death pathway (Kang et al., Free Radic Biol Med. (2019) 133:162-8). SAT1 and SAT2 regulate polyamine metabolism, a process which has long been implicated in cancer (Kang et al., Free Radic Biol Med. (2019) 133:162-8; Thomas and Thomas, J Cell Mol Med. (2003) 7:113-26). Indeed, higher expression of the FTL ferroptosis regulator, is associated with a poorer prognosis in hepatocellular carcinoma (Ke et al., Front Genet. (2022) 13:897683). Further, expression of the voltage-gated channel VDAC2 is also associated with increased risk in some cancers. VDAC2 is also a target of erastin, a small-molecule promotor of ferroptosis in cancer cells (Zhao et al., Onco Targets Ther. (2020) 13:5429-41; Yang et al., Nat Commun. (2020) 11:433). However, interestingly, expression of SAT1 as well as SAT2 has been linked to improved outcomes in several adenocarcinomas (Chang et al., Front Oncol. (2021) 11:649347; Sui et al., Pathol Int. (2021) 71:741-51; Wei et al., DNA Cell Biol. (2022) 41:116-27; Wang et al., PeerJ. (2021) 9: e11233). The association of SAT1 and SAT2 with survival individually was evaluated, but found that lower expressions of SAT1 and SAT2 individually do not correlate with survival.

TABLE 1
List of host (human) genes upregulated in the presence of bacterial
Fe-S proteins. Columns are: 1) Gene names, 2) Median z-score
in Fe-negative samples, 3) Median z-score in Fe-positive samples.
For all genes, the median z-score for Fe-positive samples was
above 0.2, and that for Fe-negative samples was below 0.

| | Median | Median |
|---|---|---|
| | z-score | z-score |
| | Fe- | Fe- |
| | negative | positive |
| Gene | samples | samples |
| GTPBP6 | −0.0277 | 0.321 |
| ABCB6 | −0.0704 | 0.4645 |
| ABHD12 | −0.1001 | 0.2765 |
| ABHD8 | −0.0417 | 0.2976 |
| ABTB1 | −0.131 | 0.3496 |
| ACOT7 | −0.1189 | 0.2467 |
| ACOT8 | −0.0817 | 0.2164 |
| ACP1 | −0.1212 | 0.2577 |
| ACSF3 | −0.1112 | 0.339 |
| ACTR3 | −0.0424 | 0.4304 |
| ADCK1 | −0.0837 | 0.3697 |
| AFMID | −0.2004 | 0.2692 |
| AGK | −0.0713 | 0.3802 |
| AHCY | −0.0195 | 0.7093 |
| AIFM1 | −0.1075 | 0.2512 |
| AIFM3 | −0.0968 | 0.5221 |
| AIG1 | −0.0457 | 0.4832 |
| AIP | −0.1796 | 0.2495 |
| AK1 | −0.1118 | 0.2961 |
| AKAP8L | −0.2984 | 0.2546 |
| AKIRIN2 | −0.1991 | 0.4706 |
| ALG5 | −0.1767 | 0.3622 |
| ALG8 | −0.0749 | 0.4738 |
| ALKBH6 | −0.0422 | 0.2119 |
| ANAPC11 | −0.1331 | 0.6095 |
| ANAPC16 | −0.0963 | 0.3871 |
| ANAPC2 | −0.0884 | 0.7782 |
| ANKRD37 | −0.1167 | 0.3227 |
| ANKRD39 | −0.1906 | 0.3357 |
| ANKRD54 | −0.0595 | 0.4334 |
| ANKRD58 | −0.0556 | 0.6985 |
| ANKZF1 | −0.0797 | 0.3001 |
| ANP32B | −0.1189 | 0.5256 |
| AP2S1 | −0.0538 | 0.4285 |
| APIP | −0.1469 | 0.2507 |
| APOA1BP | −0.1007 | 0.3934 |
| APOC2 | −0.158 | 0.4015 |
| APOO | −0.1096 | 0.2361 |
| APRT | −0.0824 | 0.3951 |
| ARF1 | −0.0995 | 0.258 |
| ARFGAP2 | −0.0353 | 0.4589 |
| ARHGAP4 | −0.2826 | 0.3168 |
| ARHGDIA | −0.0962 | 0.4877 |
| ARL8A | −0.1684 | 0.2183 |
| ARPC3 | −0.0368 | 0.5919 |
| ARPC4 | −0.1098 | 0.2399 |
| ARPC5L | −0.0597 | 0.4913 |
| ARRB2 | −0.0567 | 0.3612 |
| AS3MT | −0.051 | 0.6412 |
| ASB6 | −0.1146 | 0.4894 |
| ASF1A | −0.2859 | 0.3042 |
| ASGR1 | −0.2532 | 0.3621 |
| ASMTL | −0.1168 | 0.3796 |
| ASPSCR1 | −0.1322 | 0.2343 |
| ATF5 | −0.2764 | 0.3304 |
| ATG4D | −0.1412 | 0.4413 |
| ATG5 | −0.0565 | 0.3832 |
| ATP5C1 | −0.03 | 0.3959 |
| ATP5EP2 | −0.0409 | 0.4506 |
| ATP5G3 | −0.1488 | 0.532 |
| ATP5L | −0.0327 | 0.2221 |
| ATP6AP1 | −0.1035 | 0.4242 |
| ATP6V0B | −0.0579 | 0.2271 |
| ATP6V1E1 | −0.0576 | 0.2696 |
| ATP6V1F | −0.154 | 0.2948 |
| ATP6V1H | −0.0931 | 0.4487 |
| ATPIF1 | −0.0321 | 0.294 |
| AUH | −0.19 | 0.4758 |
| AUP1 | −0.0149 | 0.521 |
| AVPI1 | −0.1628 | 0.3053 |
| B2M | −0.05 | 0.2835 |
| B3GNTL1 | −0.0443 | 0.4052 |
| BAX | −0.1413 | 0.2285 |
| BBC3 | −0.1639 | 0.4685 |
| BCAP31 | −0.1221 | 0.6552 |
| BCAS4 | −0.1013 | 0.3186 |
| BCCIP | −0.0783 | 0.331 |
| BCL2L12 | −0.0574 | 0.238 |
| BCL3 | −0.0913 | 0.2569 |
| BID | −0.1544 | 0.5957 |
| BLOC1S3 | −0.164 | 0.5749 |
| BOLA3 | −0.1012 | 0.3498 |
| BRD4 | −0.3322 | 0.204 |
| BRD7 | −0.0355 | 0.4812 |
| BRF2 | −0.0662 | 0.2758 |
| BRMS1 | −0.1758 | 0.2678 |
| BSCL2 | −0.022 | 0.4477 |
| BTG2 | −0.2004 | 0.3902 |
| BUB3 | −0.1242 | 0.3978 |
| C10orf125 | −0.0675 | 0.5123 |
| C10orf84 | −0.0966 | 0.3445 |
| C11orf48 | −0.0872 | 0.2949 |
| C11orf51 | −0.366 | 0.3089 |
| C11orf67 | −0.2027 | 0.5315 |
| C11orf83 | −0.0701 | 0.2988 |
| C11orf84 | −0.0537 | 0.377 |
| C12orf44 | −0.1525 | 0.6619 |
| C12orf45 | −0.1715 | 0.2161 |
| C12orf47 | −0.0943 | 0.3956 |
| C12orf62 | −0.0455 | 0.4276 |
| C13orf1 | −0.1183 | 0.3216 |
| C13orf23 | −0.101 | 0.3239 |
| C13orf27 | −0.0072 | 0.515 |
| C13orf34 | −0.0692 | 0.4659 |
| C13orf37 | −0.0073 | 0.4399 |
| C14orf119 | −0.0436 | 0.3641 |
| C14orf147 | −0.0866 | 0.2369 |
| C14orf156 | −0.0931 | 0.2654 |
| C14orf166 | −0.083 | 0.3507 |
| C14orf166B | −0.5429 | 0.2208 |
| C14orf2 | −0.0451 | 0.3208 |
| C15orf24 | −0.1561 | 0.5036 |
| C15orf39 | −0.1181 | 0.4208 |
| C15orf40 | −0.0087 | 0.303 |
| C15orf57 | −0.1081 | 0.3936 |
| C15orf63 | −0.1034 | 0.3086 |
| C16orf61 | −0.1366 | 0.6109 |
| C17orf49 | −0.1759 | 0.3269 |
| C17orf61 | −0.03 | 0.3246 |
| C17orf81 | −0.0469 | 0.2051 |
| C17orf90 | −0.0918 | 0.3741 |
| C19orf42 | −0.0543 | 0.2555 |
| C19orf43 | −0.2276 | 0.2849 |
| C19orf48 | −0.0697 | 0.2693 |
| C19orf50 | −0.1692 | 0.2104 |
| C19orf53 | −0.2429 | 0.4207 |
| C19orf56 | −0.1421 | 0.2375 |
| C19orf60 | −0.1692 | 0.2177 |
| C19orf61 | −0.0123 | 0.5316 |
| C19orf66 | −0.0152 | 0.4276 |
| C19orf73 | −0.0557 | 0.3041 |
| C1GALT1C1 | −0.0057 | 0.552 |
| C1orf66 | −0.1134 | 0.3906 |
| C1QBP | −0.0113 | 0.3217 |
| C20orf111 | −0.1315 | 0.5803 |
| C20orf199 | −0.1626 | 0.578 |
| C20orf24 | −0.0428 | 0.7578 |
| C20orf4 | −0.0871 | 0.2734 |
| C20orf46 | −0.2046 | 0.5526 |
| C20orf72 | −0.0578 | 0.6427 |
| C20orf7 | −0.1915 | 0.3993 |
| C2 | −0.1568 | 0.3713 |
| C2orf79 | −0.2203 | 0.4549 |
| C6orf115 | −0.057 | 0.4369 |
| C6orf129 | −0.1782 | 0.4358 |
| C6orf35 | −0.0978 | 0.3521 |
| C7orf40 | −0.0136 | 0.5512 |
| C7orf53 | −0.0047 | 0.3 |
| C7orf54 | −0.0261 | 0.2997 |
| C7orf55 | −0.1296 | 0.2459 |
| C8orf41 | −0.0828 | 0.5487 |
| C8orf45 | −0.1102 | 0.3442 |
| C8orf55 | −0.0295 | 0.6749 |
| C8orf76 | −0.0979 | 0.3791 |
| C9orf114 | −0.2545 | 0.3261 |
| C9orf119 | −0.1177 | 0.2045 |
| C9orf140 | −0.1029 | 0.2092 |
| C9orf142 | −0.1001 | 0.3648 |
| C9orf16 | −0.1899 | 0.739 |
| C9orf23 | −0.0299 | 0.3098 |
| C9orf25 | −0.1725 | 0.4316 |
| C9orf37 | −0.1311 | 0.5172 |
| C9orf40 | −0.2317 | 0.2908 |
| C9orf6 | −0.0445 | 0.2236 |
| C9orf78 | −0.2424 | 0.4966 |
| C9orf85 | −0.1871 | 0.2337 |
| CA8 | −0.0144 | 0.6631 |
| CACNA1A | −0.0609 | 0.5624 |
| CAPNS1 | −0.0705 | 0.3589 |
| CARKD | −0.0938 | 0.5793 |
| CARS2 | −0.0917 | 0.2983 |
| CASK | −0.0503 | 0.3666 |
| CBWD2 | −0.033 | 0.2704 |
| CBWD3 | −0.0083 | 0.3633 |
| CBX4 | −0.0719 | 0.4408 |
| CBX8 | −0.0207 | 0.4549 |
| CCDC107 | −0.0616 | 0.3488 |
| CCDC124 | −0.0565 | 0.3931 |
| CCDC130 | −0.089 | 0.272 |
| CCDC137 | −0.0308 | 0.2458 |
| CCDC22 | −0.0367 | 0.3567 |
| CCDC56 | −0.0872 | 0.3993 |
| CCDC59 | −0.0879 | 0.2269 |
| CCL20 | −0.0611 | 0.3684 |
| CCNL1 | −0.1667 | 0.2995 |
| CCT7 | −0.0221 | 0.5374 |
| CD99 | −0.0346 | 0.3093 |
| CDC16 | −0.1169 | 0.2687 |
| CDK16 | −0.0081 | 0.4705 |
| CDK2AP2 | −0.0741 | 0.3364 |
| CDK5 | −0.0608 | 0.4765 |
| CDKN2D | −0.2781 | 0.254 |
| CDKN3 | −0.0534 | 0.3634 |
| CENPB | −0.1196 | 0.2461 |
| CENPM | −0.0788 | 0.409 |
| CENPW | −0.1326 | 0.4214 |
| CETN2 | −0.028 | 0.4625 |
| CHCHD2 | −0.0988 | 0.4186 |
| CHCHD3 | −0.0985 | 0.365 |
| CHCHD8 | −0.1225 | 0.3919 |
| CHMP2A | −0.017 | 0.2775 |
| CHRNA10 | −0.0031 | 0.313 |
| CHST7 | −0.2919 | 0.287 |
| CIB1 | −0.0878 | 0.2764 |
| CISD3 | −0.1486 | 0.4563 |
| CKS2 | −0.2194 | 0.5747 |
| CLEC18A | −0.0746 | 0.3685 |
| CLK3 | −0.1002 | 0.5887 |
| CLN6 | −0.1179 | 0.5461 |
| CLNS1A | −0.0242 | 0.2574 |
| CLVS1 | −0.2567 | 0.2013 |
| CMC1 | −0.1192 | 0.2563 |
| COBRA1 | −0.1109 | 0.4685 |
| COMMD1 | −0.1564 | 0.4647 |
| COMMD3 | −0.1729 | 0.5367 |
| COMMD4 | −0.1531 | 0.6482 |
| COMMD9 | −0.1378 | 0.3319 |
| COMTD1 | −0.0456 | 0.3352 |
| COPE | −0.0676 | 0.2861 |
| COQ10A | −0.0301 | 0.2915 |
| COQ3 | −0.015 | 0.431 |
| COX17 | −0.2483 | 0.2589 |
| COX4I1 | −0.1313 | 0.3386 |
| COX4NB | −0.0681 | 0.4428 |
| COX5A | −0.0503 | 0.6723 |
| COX6A1 | −0.1975 | 0.6525 |
| COX6B1 | −0.1007 | 0.3073 |
| COX6C | −0.216 | 0.3587 |
| COX7A2 | −0.1243 | 0.2504 |
| COX7B | −0.0093 | 0.6387 |
| COX8A | −0.1584 | 0.2031 |
| CREB3 | −0.1665 | 0.2538 |
| CREM | −0.1045 | 0.3976 |
| CRIPT | −0.1099 | 0.2457 |
| CRTC2 | −0.1825 | 0.3202 |
| CSK | −0.2154 | 0.3276 |
| CSNK1D | −0.0287 | 0.4269 |
| CSNK2A1 | −0.1555 | 0.323 |
| CSNK2B | −0.0169 | 0.3751 |
| CSTF3 | −0.2133 | 0.4192 |
| CTRL | −0.1915 | 0.2288 |
| CTU1 | −0.1399 | 0.3173 |
| CUEDC2 | −0.2555 | 0.4686 |
| CYB5R4 | −0.1372 | 0.3739 |
| DAP3 | −0.2091 | 0.2635 |
| DCTN6 | −0.1117 | 0.5517 |
| DCXR | −0.1151 | 0.39 |
| DDA1 | −0.0903 | 0.2614 |
| DDRGK1 | −0.0656 | 0.5348 |
| DDX27 | −0.0532 | 0.6369 |
| DDX39 | −0.1615 | 0.4827 |
| DEDD2 | −0.1155 | 0.3586 |
| DENND1A | −0.1575 | 0.5476 |
| DHRSX | −0.2457 | 0.5302 |
| DIABLO | −0.0616 | 0.2557 |
| DKC1 | −0.0784 | 0.3387 |
| DLEU2 | −0.1627 | 0.4685 |
| DNAJA1 | −0.0346 | 0.2456 |
| DNAJB11 | −0.0117 | 0.5858 |
| DNAJB12 | −0.1297 | 0.2316 |
| DNAJC15 | −0.0141 | 0.5646 |
| DNAJC25 | −0.0651 | 0.3915 |
| DNM1 | −0.1087 | 0.4886 |
| DNTTIP1 | −0.0638 | 0.5947 |
| DOLK | −0.1802 | 0.292 |
| DOLPP1 | −0.0487 | 0.2991 |
| DPM1 | −0.0196 | 0.3857 |
| DPM2 | −0.1821 | 0.571 |
| DPM3 | −0.201 | 0.3081 |
| DPP7 | −0.1791 | 0.7331 |
| DRAM2 | −0.1502 | 0.3903 |
| DRG1 | −0.1371 | 0.2649 |
| DSCR6 | −0.0187 | 0.5105 |
| DUS1L | −0.1162 | 0.4867 |
| DUSP2 | −0.1661 | 0.2992 |
| DYNLRB1 | −0.1141 | 0.6251 |
| DYNLT1 | −0.0875 | 0.3886 |
| EBP | −0.1099 | 0.6596 |
| EBPL | −0.1633 | 0.5702 |
| ECE2 | −0.0351 | 0.5937 |
| ECHS1 | −0.0702 | 0.3412 |
| EDF1 | −0.2103 | 0.5597 |
| EFHA1 | −0.1023 | 0.2924 |
| EFNA1 | −0.2754 | 0.6259 |
| EIF2B4 | −0.0325 | 0.4885 |
| EIF2S2 | −0.0983 | 0.3211 |
| EIF3J | −0.1121 | 0.4187 |
| EIF3K | −0.073 | 0.4291 |
| EIF3M | −0.0848 | 0.2651 |
| EIF4EBP1 | −0.2068 | 0.5816 |
| EIF5A | −0.0265 | 0.4054 |
| ELOF1 | −0.0647 | 0.2897 |
| EMD | −0.215 | 0.3469 |
| ENDOG | −0.0686 | 0.2259 |
| EPS8L3 | −0.3884 | 0.8468 |
| ERGIC3 | −0.0048 | 0.568 |
| ERP29 | −0.2396 | 0.3355 |
| ERP44 | −0.0495 | 0.4313 |
| ESYT3 | −0.0499 | 0.215 |
| ETFA | −0.0546 | 0.6163 |
| EWSR1 | −0.1229 | 0.271 |
| EXD3 | −0.1034 | 0.7508 |
| EXOSC1 | −0.1355 | 0.4596 |
| EXOSC8 | −0.1003 | 0.4539 |
| EZH2 | −0.0636 | 0.5123 |
| F8A1 | −0.1575 | 0.6094 |
| FAM100A | −0.1362 | 0.5203 |
| FAM100B | −0.1577 | 0.4649 |
| FAM125A | −0.0897 | 0.4047 |
| FAM125B | −0.1021 | 0.2608 |
| FAM136A | −0.0599 | 0.3239 |
| FAM158A | −0.1016 | 0.4038 |
| FAM167B | −0.166 | 0.3001 |
| FAM192A | −0.0914 | 0.4454 |
| FAM3A | −0.0977 | 0.5431 |
| FAM43A | −0.0869 | 0.332 |
| FAM45A | −0.047 | 0.3702 |
| FAM50A | −0.1077 | 0.6785 |
| FAM58A | −0.159 | 0.562 |
| FAM73B | −0.1001 | 0.6477 |
| FAM82A2 | −0.1214 | 0.5492 |
| FAM96A | −0.0481 | 0.4389 |
| FAM96B | −0.247 | 0.2264 |
| FARSA | −0.0302 | 0.3459 |
| FASTK | −0.0311 | 0.3086 |
| FASTKD5 | −0.024 | 0.6001 |
| FAU | −0.1902 | 0.2262 |
| FBXL12 | −0.1038 | 0.3912 |
| FBXL15 | −0.0634 | 0.3955 |
| FBXO33 | −0.0845 | 0.4004 |
| FFAR2 | −0.156 | 0.4379 |
| FGFBP3 | −0.011 | 0.4189 |
| FITM1 | −0.0637 | 0.37 |
| FITM2 | −0.0976 | 0.2254 |
| FKBP1A | −0.1088 | 0.5194 |
| FKBP2 | −0.2018 | 0.3378 |
| FN3KRP | −0.0243 | 0.6059 |
| FNBP4 | −0.0844 | 0.2302 |
| FRAT1 | −0.1124 | 0.3186 |
| FRAT2 | −0.021 | 0.4864 |
| FSCN3 | −0.023 | 0.5842 |
| FTL | −0.1313 | 0.2732 |
| FXN | −0.2178 | 0.3982 |
| GABARAP | −0.1164 | 0.4765 |
| GADD45G | −0.0673 | 0.2606 |
| GCH1 | −0.0799 | 0.4262 |
| GDI1 | −0.1794 | 0.4174 |
| GEMIN7 | −0.0341 | 0.4612 |
| GFI1 | −0.0917 | 0.3763 |
| GGCT | −0.0524 | 0.4497 |
| GHITM | −0.0649 | 0.3697 |
| GK | −0.0233 | 0.2662 |
| GLA | −0.2597 | 0.3178 |
| GLRX2 | −0.0502 | 0.442 |
| GLRX | −0.0915 | 0.3798 |
| GLRX3 | −0.132 | 0.3281 |
| GMIP | −0.0756 | 0.3457 |
| GPI | −0.1861 | 0.4664 |
| GPKOW | −0.1819 | 0.4283 |
| GPR37L1 | −0.0371 | 0.6136 |
| GPS1 | −0.1299 | 0.4316 |
| GPS2 | −0.0099 | 0.3193 |
| GRINA | −0.0554 | 0.5609 |
| GSTM4 | −0.125 | 0.3844 |
| GSTO1 | −0.1192 | 0.5939 |
| GTF2A2 | −0.1353 | 0.4626 |
| GTF2F2 | −0.1168 | 0.4126 |
| GTF3A | −0.129 | 0.371 |
| H1FX | −0.224 | 0.2528 |
| H3F3A | −0.1228 | 0.2373 |
| HAGH | −0.2977 | 0.5226 |
| HAUS7 | −0.1048 | 0.6634 |
| HAUS8 | −0.1313 | 0.2524 |
| HDAC2 | −0.0671 | 0.5611 |
| HDDC3 | −0.1306 | 0.3102 |
| HDGF | −0.0454 | 0.4864 |
| HES1 | −0.0714 | 0.3821 |
| HEXA | −0.1541 | 0.2878 |
| HIGD1A | −0.1139 | 0.2479 |
| HM13 | −0.0399 | 0.5407 |
| HMBS | −0.0498 | 0.4547 |
| HMGB1 | −0.0441 | 0.3528 |
| HMGB3 | −0.1517 | 0.4814 |
| HMP19 | −0.356 | 0.2004 |
| HN1 | −0.0776 | 0.2566 |
| HNRNPA3P1 | −0.0651 | 0.4781 |
| HNRNPL | −0.1066 | 0.2763 |
| HNRPDL | −0.0343 | 0.3596 |
| HPCAL1 | −0.007 | 0.4392 |
| HPRT1 | −0.0379 | 0.5032 |
| HS3ST5 | −0.4726 | 0.6617 |
| HSBP1 | −0.0466 | 0.5207 |
| HSD17B10 | −0.0508 | 0.3766 |
| HSD17B14 | −0.0625 | 0.5871 |
| HSF2 | −0.1331 | 0.2513 |
| HSP90AB4P | −0.2982 | 0.3208 |
| HSPE1 | −0.0246 | 0.5192 |
| ICT1 | −0.0704 | 0.4199 |
| IDH2 | −0.1405 | 0.2311 |
| IDH3A | −0.0973 | 0.6779 |
| IDH3G | −0.1755 | 0.6694 |
| IER2 | −0.0641 | 0.6008 |
| IER5L | −0.0634 | 0.2745 |
| IFI30 | −0.1077 | 0.5953 |
| IFITM1 | −0.0536 | 0.412 |
| IGBP1 | −0.1062 | 0.3794 |
| IKBKG | −0.1289 | 0.5159 |
| ILF2 | −0.0727 | 0.4935 |
| ING1 | −0.0906 | 0.3282 |
| IRF2BP1 | −0.0041 | 0.2307 |
| IRF3 | −0.0681 | 0.3265 |
| ISG15 | −0.1581 | 0.4307 |
| ISG20 | −0.0363 | 0.2761 |
| ITPA | −0.0116 | 0.5347 |
| JMJD6 | −0.2118 | 0.4873 |
| JTB | −0.1398 | 0.403 |
| KARS | −0.0535 | 0.4063 |
| KATNA1 | −0.1215 | 0.2348 |
| KCNJ2 | −0.13 | 0.3969 |
| KCNMB3 | −0.0999 | 0.428 |
| KCTD17 | −0.0066 | 0.2901 |
| KDELR1 | −0.0478 | 0.4897 |
| KHK | −0.0479 | 0.5671 |
| KIAA1279 | −0.075 | 0.5908 |
| KIAA1598 | −0.1961 | 0.2873 |
| KIF21B | −0.0544 | 0.4357 |
| KLRB1 | −0.0094 | 0.4164 |
| KRTCAP2 | −0.1649 | 0.3575 |
| LAGE3 | −0.1488 | 0.2832 |
| LAS1L | −0.1595 | 0.4621 |
| LENG1 | −0.0128 | 0.2909 |
| LEPROTL1 | −0.1165 | 0.2028 |
| LGALS3BP | −0.0258 | 0.2721 |
| LIG1 | −0.0345 | 0.2923 |
| LIMD2 | −0.1405 | 0.2185 |
| LIN37 | −0.1767 | 0.3086 |
| LINC01003 | −0.0879 | 0.3391 |
| LOC100133331 | −0.0367 | 0.2911 |
| LOC100133985 | −0.1155 | 0.2638 |
| LOC143188 | −0.052 | 0.4275 |
| CUTALP | −0.0681 | 0.3184 |
| LOC388789 | −0.0005 | 0.475 |
| SNHG17 | −0.0199 | 0.6421 |
| PTGES2-AS1 | −0.0275 | 0.2098 |
| LINC03025 | −0.1877 | 0.4525 |
| LOC606724 | −0.2118 | 0.2909 |
| SPCS2P4 | −0.2195 | 0.2938 |
| LOC728743 | −0.1442 | 0.283 |
| PIN4P1 | −0.0689 | 0.2371 |
| GTF2IRD1P1 | −0.1707 | 0.4332 |
| LRRC16B | −0.1141 | 0.3944 |
| LRRC37A3 | −0.0328 | 0.3919 |
| LRRC43 | −0.3013 | 0.2564 |
| LRRC45 | −0.0823 | 0.3918 |
| LSM1 | −0.1055 | 0.4266 |
| LSM4 | −0.195 | 0.3121 |
| LSM5 | −0.0871 | 0.4518 |
| LSM7 | −0.1368 | 0.2643 |
| LTBR | −0.0721 | 0.2453 |
| LY6G5C | −0.1067 | 0.2634 |
| LYRM1 | −0.1507 | 0.3397 |
| MAFG | −0.1668 | 0.5137 |
| MAGED4 | −0.1166 | 0.3503 |
| MAGEF1 | −0.0903 | 0.4258 |
| MANF | −0.0633 | 0.5382 |
| MAP1LC3A | −0.1401 | 0.5928 |
| MAP1LC3B2 | −0.2344 | 0.3954 |
| MAP1LC3B | −0.1128 | 0.4459 |
| MAP2K1 | −0.1716 | 0.3057 |
| MAP3K8 | −0.0444 | 0.5851 |
| 5-Mar | −0.1128 | 0.2731 |
| MCAT | −0.0467 | 0.3497 |
| MCRS1 | −0.1145 | 0.4011 |
| MCTS1 | −0.0534 | 0.3156 |
| MDK | −0.0194 | 0.3084 |
| MEA1 | −0.1866 | 0.3545 |
| MED22 | −0.1074 | 0.4074 |
| MED27 | −0.2021 | 0.2682 |
| MED4 | −0.1499 | 0.4172 |
| MESP1 | −0.1046 | 0.6135 |
| MESP2 | −0.1255 | 0.364 |
| METRNL | −0.1044 | 0.4157 |
| METTL11A | −0.1792 | 0.4106 |
| MFSD6L | −0.2439 | 0.3768 |
| MGC70857 | −0.0471 | 0.2603 |
| MID1IP1 | −0.1622 | 0.3737 |
| MKKS | −0.0454 | 0.3614 |
| MORF4L2 | −0.0126 | 0.4635 |
| MORN2 | −0.0614 | 0.5024 |
| MPDU1 | −0.0866 | 0.4157 |
| MPP1 | −0.1087 | 0.3387 |
| MRP63 | −0.205 | 0.387 |
| MRPL12 | −0.0587 | 0.6449 |
| MRPL15 | −0.1582 | 0.3483 |
| MRPL18 | −0.1161 | 0.3877 |
| MRPL34 | −0.1566 | 0.4933 |
| MRPL36 | −0.0827 | 0.2449 |
| MRPL38 | −0.09 | 0.4884 |
| MRPL41 | −0.0747 | 0.6925 |
| MRPL4 | −0.1221 | 0.3555 |
| MRPL47 | −0.0444 | 0.2744 |
| MRPL48 | −0.1523 | 0.4574 |
| MRPL50 | −0.0043 | 0.3628 |
| MRPL52 | −0.1278 | 0.2702 |
| MRPL54 | −0.2203 | 0.3832 |
| MRPL9 | −0.1091 | 0.3616 |
| MRPS11 | −0.088 | 0.4666 |
| MRPS12 | −0.073 | 0.3846 |
| MRPS21 | −0.0406 | 0.2032 |
| MRPS2 | −0.1669 | 0.5493 |
| MRPS26 | −0.0419 | 0.6909 |
| MRPS33 | −0.0993 | 0.429 |
| MSI1 | −0.1057 | 0.5926 |
| MST1P2 | −0.0922 | 0.2301 |
| MST1P9 | −0.0755 | 0.2742 |
| MSX1 | −0.0927 | 0.5815 |
| MTCH2 | −0.0388 | 0.5194 |
| MTFMT | −0.0361 | 0.7181 |
| MTHFD2 | −0.1754 | 0.2475 |
| MTHFS | −0.1922 | 0.406 |
| MTIF3 | −0.1534 | 0.384 |
| MTP18 | −0.0705 | 0.4108 |
| MXD3 | −0.0886 | 0.4469 |
| MYEOV2 | −0.1715 | 0.411 |
| MYL12B | −0.0235 | 0.3842 |
| MYLK2 | −0.2295 | 0.387 |
| NAA10 | −0.1608 | 0.563 |
| NAA20 | −0.1286 | 0.4203 |
| LSM8 | −0.1506 | 0.2946 |
| NAALADL1 | −0.1384 | 0.4434 |
| NAE1 | −0.0612 | 0.4586 |
| NANS | −0.1365 | 0.4719 |
| NARF | −0.1556 | 0.8787 |
| NARS2 | −0.0639 | 0.5028 |
| NCOA7 | −0.1572 | 0.4288 |
| NCRNA00081 | −0.1894 | 0.2874 |
| NCRNA00116 | −0.0246 | 0.3902 |
| NDNL2 | −0.2924 | 0.4722 |
| NDOR1 | −0.1083 | 0.3795 |
| NDUFA13 | −0.1918 | 0.5237 |
| NDUFA1 | −0.1837 | 0.4681 |
| NDUFA2 | −0.0283 | 0.551 |
| NDUFA3 | −0.138 | 0.3416 |
| NDUFA4 | −0.0704 | 0.2764 |
| NDUFA8 | −0.1974 | 0.2922 |
| NDUFAF1 | −0.0186 | 0.2021 |
| NDUFAF4 | −0.0697 | 0.326 |
| NDUFB11 | −0.1074 | 0.3978 |
| NDUFB2 | −0.0665 | 0.4577 |
| NDUFB3 | −0.0928 | 0.3762 |
| NDUFB4 | −0.1442 | 0.2645 |
| NDUFB8 | −0.1229 | 0.5067 |
| NDUFB9 | −0.135 | 0.3445 |
| NDUFC2 | −0.1864 | 0.484 |
| NDUFS2 | −0.0751 | 0.3113 |
| NDUFS3 | −0.1729 | 0.411 |
| NDUFS6 | −0.09 | 0.391 |
| NECAB3 | −0.0135 | 0.412 |
| NELF | −0.0691 | 0.556 |
| NENF | −0.1943 | 0.3023 |
| NEURL | −0.2548 | 0.4022 |
| NFIL3 | −0.1975 | 0.3189 |
| NFKBIB | −0.0499 | 0.3779 |
| NFKBID | −0.1719 | 0.3524 |
| NFS1 | −0.0882 | 0.3504 |
| NINJ2 | −0.0235 | 0.3299 |
| NKAP | −0.1182 | 0.3632 |
| NME2 | −0.1432 | 0.2521 |
| NME2P1 | −0.1186 | 0.3955 |
| NMRAL1 | −0.0972 | 0.3938 |
| NONO | −0.0514 | 0.2256 |
| NOP10 | −0.0336 | 0.366 |
| NOP56 | −0.0619 | 0.4379 |
| NOSIP | −0.1261 | 0.6358 |
| NR1H2 | −0.1435 | 0.4746 |
| NR2C2AP | −0.2798 | 0.3191 |
| NRL | −0.0191 | 0.3316 |
| NSDHL | −0.0964 | 0.4232 |
| NSFL1C | −0.0434 | 0.2779 |
| NSMCE4A | −0.0231 | 0.4023 |
| NT5C3 | −0.0234 | 0.3901 |
| NT5C3L | −0.0646 | 0.3693 |
| NUCB1 | −0.1739 | 0.432 |
| NUDT1 | −0.0854 | 0.3226 |
| NUDT19 | −0.0805 | 0.4075 |
| NUDT22 | −0.1316 | 0.2043 |
| NUDT5 | −0.0476 | 0.611 |
| NUDT8 | −0.129 | 0.3268 |
| NUTF2 | −0.0867 | 0.4639 |
| NXT1 | −0.1496 | 0.2496 |
| ODF2 | −0.0854 | 0.402 |
| OLA1 | −0.066 | 0.4313 |
| ORMDL1 | −0.1347 | 0.288 |
| OSM | −0.1119 | 0.4371 |
| OST4 | −0.2397 | 0.515 |
| OTOF | −0.2652 | 0.2582 |
| OTUD5 | −0.003 | 0.4977 |
| OXCT2 | −0.1922 | 0.2494 |
| OXT | −0.2863 | 0.4346 |
| PAF1 | −0.1169 | 0.38 |
| PAFAH1B3 | −0.0596 | 0.3337 |
| PANK2 | −0.0087 | 0.4068 |
| PARK7 | −0.0936 | 0.2405 |
| PARP16 | −0.1451 | 0.2179 |
| PAX6 | −0.1031 | 0.232 |
| PCBD1 | −0.0337 | 0.3427 |
| PCGF6 | −0.1173 | 0.4795 |
| PCID2 | −0.1482 | 0.2469 |
| PCK2 | −0.1956 | 0.2275 |
| PCNA | −0.0857 | 0.381 |
| PCYT2 | −0.0671 | 0.3955 |
| PDCL3 | −0.1347 | 0.4242 |
| PDHA1 | −0.1068 | 0.4226 |
| PDHX | −0.1498 | 0.3276 |
| PDRG1 | −0.1092 | 0.3531 |
| PDZD11 | −0.1237 | 0.5771 |
| PEBP1 | −0.0875 | 0.3664 |
| PEX16 | −0.2348 | 0.5204 |
| PEX7 | −0.1088 | 0.2771 |
| PFDN4 | −0.0613 | 0.6873 |
| PFKFB1 | −0.0084 | 0.364 |
| PHF11 | −0.0791 | 0.3966 |
| PHPT1 | −0.1253 | 0.5436 |
| PIGA | −0.1341 | 0.42 |
| PIGB | −0.0191 | 0.4203 |
| PIGF | −0.0442 | 0.705 |
| PIGZ | −0.0721 | 0.4757 |
| PIM2 | −0.1917 | 0.2785 |
| PIM3 | −0.1523 | 0.5378 |
| PIPOX | −0.2255 | 0.3503 |
| PIPSL | −0.1447 | 0.4281 |
| PIR | −0.1696 | 0.4561 |
| PLA2G4C | −0.2045 | 0.3795 |
| PLEKHJ1 | −0.0564 | 0.2943 |
| PLIN2 | −0.0126 | 0.4323 |
| PMAIP1 | −0.0444 | 0.564 |
| PMF1 | −0.1313 | 0.3094 |
| PNN | −0.041 | 0.4594 |
| PNRC1 | −0.1999 | 0.2889 |
| POLR1D | −0.0748 | 0.3505 |
| POLR2F | −0.1805 | 0.3435 |
| POLR2H | −0.0336 | 0.5578 |
| POLR2I | −0.1271 | 0.2422 |
| POLR3F | −0.1169 | 0.4245 |
| POLR3K | −0.0796 | 0.5425 |
| POMP | −0.0546 | 0.5994 |
| POU2AF1 | −0.0817 | 0.5753 |
| POU2F1 | −0.097 | 0.3162 |
| PPA1 | −0.1738 | 0.3564 |
| PPCDC | −0.0216 | 0.4301 |
| PPIA | −0.0402 | 0.3547 |
| PPIAL4C | −0.1827 | 0.555 |
| PPIB | −0.1006 | 0.4274 |
| PPIF | −0.1322 | 0.6782 |
| PPP1R2 | −0.1579 | 0.5327 |
| PPP2R2D | −0.0102 | 0.3755 |
| PPP2R3B | −0.0873 | 0.379 |
| PQBP1 | −0.094 | 0.4067 |
| PRAF2 | −0.1147 | 0.3857 |
| PRCC | −0.0837 | 0.447 |
| PRDX2 | −0.0762 | 0.3466 |
| PRDX4 | −0.2105 | 0.3135 |
| PREB | −0.1352 | 0.4029 |
| PRELID1 | −0.1008 | 0.292 |
| PREP | −0.1139 | 0.3659 |
| PRKRA | −0.1687 | 0.3152 |
| ProSAPiP1 | −0.0917 | 0.5912 |
| PRR5 | −0.1336 | 0.4241 |
| PRR5L | −0.0573 | 0.5616 |
| PSENEN | −0.2152 | 0.2844 |
| PSMA1 | −0.0668 | 0.5921 |
| PSMA2 | −0.177 | 0.3682 |
| PSMA3 | −0.0643 | 0.3111 |
| PSMA4 | −0.0712 | 0.5887 |
| PSMA5 | −0.0549 | 0.5403 |
| PSMA7 | −0.1172 | 0.3643 |
| PSMB1 | −0.1356 | 0.6811 |
| PSMB3 | −0.174 | 0.3743 |
| PSMB4 | −0.2356 | 0.2799 |
| PSMB5 | −0.078 | 0.2454 |
| PSMB7 | −0.2237 | 0.2156 |
| PSMC1 | −0.0467 | 0.352 |
| PSMC3 | −0.192 | 0.4789 |
| PSMC4 | −0.1465 | 0.5316 |
| PSMC6 | −0.0434 | 0.4491 |
| PSMD10 | −0.0554 | 0.283 |
| PSMD14 | −0.0741 | 0.4118 |
| PSMD4 | −0.1266 | 0.689 |
| PSMD6 | −0.1501 | 0.3585 |
| PSMD7 | −0.0763 | 0.67 |
| PSMD8 | −0.068 | 0.2049 |
| PSME1 | −0.1386 | 0.245 |
| PSME2 | −0.0947 | 0.3909 |
| PSMG2 | −0.102 | 0.7347 |
| PSMG3 | −0.0124 | 0.4595 |
| PTGES2 | −0.2426 | 0.63 |
| PTPMT1 | −0.102 | 0.5414 |
| PTPRA | −0.1357 | 0.242 |
| PTRH1 | −0.1573 | 0.2377 |
| PTS | −0.1648 | 0.7095 |
| PVRL2 | −0.0188 | 0.2024 |
| PYCR1 | −0.0605 | 0.5569 |
| RAB15 | −0.033 | 0.5026 |
| RAB3A | −0.2008 | 0.3417 |
| RAB40B | −0.1194 | 0.4485 |
| RAB4B | −0.0161 | 0.2636 |
| RAB8A | −0.2165 | 0.3881 |
| RAB9A | −0.0705 | 0.3964 |
| RABAC1 | −0.1342 | 0.4455 |
| RAD9A | −0.1384 | 0.4779 |
| RALY | −0.003 | 0.555 |
| RANGRF | −0.148 | 0.4496 |
| RASSF4 | −0.0598 | 0.3855 |
| RBM42 | −0.1744 | 0.2443 |
| RBMX2 | −0.2194 | 0.4264 |
| RBMX | −0.089 | 0.2283 |
| RBX1 | −0.0899 | 0.3909 |
| RCN1 | −0.0586 | 0.5314 |
| RCN2 | −0.1893 | 0.5053 |
| RELT | −0.1652 | 0.5018 |
| REXO4 | −0.0844 | 0.4489 |
| RFK | −0.0566 | 0.575 |
| RFNG | −0.0427 | 0.4481 |
| RFXAP | −0.0637 | 0.3566 |
| RHBDD2 | −0.2519 | 0.4056 |
| RHEB | −0.1921 | 0.3469 |
| RILPL2 | −0.1787 | 0.3548 |
| RLN1 | −0.3029 | 0.3398 |
| RNASEH2B | −0.1945 | 0.4343 |
| RNASEK | −0.1428 | 0.3809 |
| RNF113A | −0.1113 | 0.5945 |
| RNF114 | −0.0604 | 0.725 |
| RNF181 | −0.0887 | 0.2939 |
| RNF5 | −0.1042 | 0.3099 |
| ROBLD3 | −0.1468 | 0.4642 |
| ROMO1 | −0.0579 | 0.3688 |
| RP9 | −0.0925 | 0.5572 |
| RPAIN | −0.1183 | 0.5142 |
| RPL10 | −0.1899 | 0.2511 |
| RPL13A | −0.0751 | 0.2546 |
| RPL18 | −0.0852 | 0.3232 |
| RPL18A | −0.1463 | 0.581 |
| RPL23 | −0.232 | 0.3164 |
| RPL23A | −0.0258 | 0.2588 |
| RPL23P8 | −0.1841 | 0.6089 |
| RPL24 | −0.1319 | 0.344 |
| RPL27A | −0.1284 | 0.3689 |
| RPL28 | −0.1283 | 0.2372 |
| RPL35 | −0.1407 | 0.3195 |
| RPL35A | −0.096 | 0.503 |
| RPL37 | −0.1134 | 0.3078 |
| RPL38 | −0.1842 | 0.2252 |
| RPL39 | −0.2497 | 0.8195 |
| RPL4 | −0.0432 | 0.2777 |
| RPL7A | −0.1079 | 0.3548 |
| RPLP1 | −0.0667 | 0.4482 |
| RPLP2 | −0.0318 | 0.3583 |
| RPPH1 | −0.1653 | 0.3921 |
| RPS11 | −0.1729 | 0.4373 |
| RPS13 | −0.1233 | 0.448 |
| RPS16 | −0.1585 | 0.3076 |
| RPS17 | −0.1456 | 0.243 |
| RPS19 | −0.0915 | 0.3186 |
| RPS20 | −0.2594 | 0.5044 |
| RPS24 | −0.1314 | 0.3201 |
| RPS27 | −0.0759 | 0.4251 |
| RPS27L | −0.0678 | 0.5085 |
| RPS29 | −0.0951 | 0.2123 |
| RSL24D1 | −0.135 | 0.4 |
| RUVBL2 | −0.1313 | 0.4436 |
| RWDD1 | −0.1484 | 0.5996 |
| SAA4 | −0.1253 | 0.2098 |
| SAP18 | −0.2584 | 0.6181 |
| SAT1 | −0.1437 | 0.4296 |
| SAT2 | −0.1921 | 0.5128 |
| SCAMP2 | −0.1635 | 0.2734 |
| SCAND1 | −0.0806 | 0.4945 |
| SCNM1 | −0.1567 | 0.3137 |
| SDHAF1 | −0.0392 | 0.3557 |
| SEC11A | −0.0709 | 0.3414 |
| SEC61B | −0.2773 | 0.3551 |
| SECTM1 | −0.027 | 0.5629 |
| SELK | −0.119 | 0.6059 |
| SELO | −0.1107 | 0.3029 |
| SELS | −0.2257 | 0.6497 |
| SERF2 | −0.1864 | 0.3038 |
| SERP1 | −0.0805 | 0.3808 |
| SET | −0.1108 | 0.2254 |
| SF3B6 | −0.1485 | 0.452 |
| SF3B4 | −0.1983 | 0.3304 |
| SF3B5 | −0.1532 | 0.3592 |
| SF4 | −0.2202 | 0.2498 |
| SFT2D1 | −0.0268 | 0.3755 |
| SH3GLB2 | −0.128 | 0.336 |
| SLC25A19 | −0.1183 | 0.2573 |
| SLC25A29 | −0.0637 | 0.4568 |
| SLC25A38 | −0.1225 | 0.3073 |
| SLC25A5 | −0.0509 | 0.6484 |
| SLC25A6 | −0.2003 | 0.312 |
| SLC2A8 | −0.2068 | 0.3013 |
| SLC35D2 | −0.0362 | 0.6388 |
| SLC35E4 | −0.1129 | 0.2988 |
| SLCO3A1 | −0.0831 | 0.358 |
| SLTM | −0.1098 | 0.3744 |
| SMOX | −0.0475 | 0.3084 |
| SMPD2 | −0.044 | 0.5423 |
| SMS | −0.0212 | 0.532 |
| SNAPC4 | −0.0617 | 0.366 |
| SNHG11 | −0.0547 | 0.3111 |
| SNHG7 | −0.0875 | 0.2673 |
| SNORD17 | −0.1263 | 0.4047 |
| SNRNP25 | −0.1469 | 0.6409 |
| SNRPA1 | −0.1818 | 0.2258 |
| SNRPB2 | −0.0173 | 0.5673 |
| SNRPB | −0.1385 | 0.5195 |
| SNRPD2 | −0.1137 | 0.229 |
| SNRPF | −0.1904 | 0.3568 |
| SNRPG | −0.0829 | 0.4389 |
| SNX22 | −0.0798 | 0.4429 |
| SNX3 | −0.0482 | 0.4363 |
| SPATA2L | −0.0104 | 0.5949 |
| SPCS1 | −0.0248 | 0.3067 |
| SPCS2 | −0.1764 | 0.3711 |
| SPG21 | −0.2469 | 0.5204 |
| SRP14 | −0.1108 | 0.4638 |
| SS18L2 | −0.0671 | 0.6801 |
| SSBP1 | −0.1177 | 0.2915 |
| SSNA1 | −0.2363 | 0.3498 |
| SSR2 | −0.1475 | 0.6017 |
| SSR4 | −0.2118 | 0.5431 |
| ST7 | −0.1038 | 0.3273 |
| STIP1 | −0.0771 | 0.4378 |
| STRA13 | −0.0432 | 0.4621 |
| STRBP | −0.0802 | 0.2963 |
| STX5 | −0.0829 | 0.3106 |
| SUGT1 | −0.133 | 0.4833 |
| SURF1 | −0.1114 | 0.2309 |
| SURF2 | −0.1546 | 0.4483 |
| SURF4 | −0.0929 | 0.6616 |
| SURF6 | −0.1373 | 0.7425 |
| SYNGR2 | −0.0139 | 0.2347 |
| SYNGR3 | −0.1982 | 0.2463 |
| SYS1 | −0.0878 | 0.2504 |
| TALDO1 | −0.2501 | 0.3091 |
| TARS | −0.109 | 0.386 |
| TAZ | −0.103 | 0.6415 |
| TBC1D20 | −0.047 | 0.4199 |
| TBCD | −0.1549 | 0.2557 |
| TBPL1 | −0.0918 | 0.5263 |
| TCEB1 | −0.1644 | 0.3546 |
| TCEB2 | −0.05 | 0.2216 |
| TDP2 | −0.3915 | 0.3057 |
| TERF2IP | −0.1098 | 0.3946 |
| TEX19 | −0.2748 | 0.3137 |
| TEX261 | −0.0638 | 0.327 |
| TFE3 | −0.0711 | 0.2955 |
| TFPT | −0.173 | 0.4679 |
| TGDS | −0.0467 | 0.375 |
| TGIF1 | −0.0106 | 0.435 |
| THAP3 | −0.1316 | 0.3132 |
| THOC4 | −0.0221 | 0.3582 |
| TIGD3 | −0.1145 | 0.3787 |
| TIMM16 | −0.1643 | 0.2698 |
| TIMM17B | −0.23 | 0.3879 |
| TIMM50 | −0.1061 | 0.2643 |
| TM2D2 | −0.1057 | 0.3752 |
| TM2D3 | −0.0856 | 0.2155 |
| TMED1 | −0.0329 | 0.5363 |
| TMEM111 | −0.0582 | 0.2992 |
| TMEM11 | −0.1214 | 0.281 |
| TMEM126A | −0.0466 | 0.2616 |
| TMEM147 | −0.1336 | 0.2922 |
| TMEM160 | −0.1018 | 0.395 |
| TMEM163 | −0.1252 | 0.2302 |
| TMEM176A | −0.065 | 0.2846 |
| TMEM183A | −0.0762 | 0.5018 |
| TMEM187 | −0.0537 | 0.7013 |
| TMEM198 | −0.016 | 0.4394 |
| TMEM208 | −0.0598 | 0.5673 |
| TMEM214 | −0.0908 | 0.5019 |
| TMEM216 | −0.108 | 0.3184 |
| TMEM44 | −0.1537 | 0.7739 |
| TMEM70 | −0.0803 | 0.4389 |
| TMEM85 | −0.1352 | 0.4881 |
| TMEM93 | −0.1324 | 0.2472 |
| TMSL3 | −0.1068 | 0.2955 |
| TMUB1 | −0.0931 | 0.2294 |
| TMX2 | −0.0556 | 0.28 |
| TNNC2 | −0.1961 | 0.3657 |
| TOR1A | −0.1976 | 0.3453 |
| TOR1B | −0.0604 | 0.565 |
| TOR2A | −0.2183 | 0.542 |
| TP53I13 | −0.0444 | 0.3982 |
| TP53RK | −0.0863 | 0.3289 |
| TPRA1 | −0.1192 | 0.5597 |
| TPRKB | −0.105 | 0.2519 |
| TPRN | −0.1473 | 0.4836 |
| TPT1 | −0.1834 | 0.4007 |
| TRAF2 | −0.0617 | 0.2094 |
| TREML3 | −0.4637 | 0.2239 |
| TREX1 | −0.1052 | 0.3608 |
| TRIB3 | −0.1382 | 0.2406 |
| TRIM11 | −0.0388 | 0.3615 |
| TRMT2B | −0.0899 | 0.3382 |
| TRMT6 | −0.0677 | 0.2347 |
| TRPT1 | −0.152 | 0.3318 |
| TSEN34 | −0.1805 | 0.3335 |
| TSPAN33 | −0.1095 | 0.3078 |
| TSR2 | −0.0164 | 0.5989 |
| TSSC1 | −0.0724 | 0.3875 |
| TTC32 | −0.1338 | 0.2955 |
| TTF1 | −0.1125 | 0.3051 |
| TUBB2C | −0.0787 | 0.558 |
| TXNRD1 | −0.0875 | 0.4186 |
| UBA52 | −0.1986 | 0.2159 |
| UBB | −0.1214 | 0.5259 |
| UBE2J1 | −0.0754 | 0.3975 |
| UBE2N | −0.0804 | 0.3901 |
| UBE2V1 | −0.0475 | 0.2768 |
| UBL4A | −0.1147 | 0.2658 |
| UBL5 | −0.1389 | 0.3045 |
| UBXN1 | −0.1064 | 0.3133 |
| UCK1 | −0.1516 | 0.3385 |
| UCP2 | −0.0796 | 0.4594 |
| UGT1A3 | −0.1891 | 0.2249 |
| UPF3B | −0.0397 | 0.3747 |
| UQCR10 | −0.0214 | 0.3375 |
| UQCRC1 | −0.2193 | 0.278 |
| URM1 | −0.1261 | 0.5039 |
| USE1 | −0.1554 | 0.3707 |
| USF1 | −0.1821 | 0.253 |
| USP20 | −0.0949 | 0.2383 |
| UXT | −0.2321 | 0.3963 |
| VBP1 | −0.1792 | 0.3999 |
| VPS16 | −0.0748 | 0.4963 |
| VPS29 | −0.0115 | 0.291 |
| WASH3P | −0.1612 | 0.4437 |
| WASH5P | −0.0274 | 0.749 |
| WASH7P | −0.2003 | 0.4888 |
| WBP4 | −0.083 | 0.2896 |
| WBSCR22 | −0.0623 | 0.4482 |
| WBSCR28 | −0.1416 | 0.5223 |
| WDR45 | −0.1253 | 0.5754 |
| WDR85 | −0.1048 | 0.8653 |
| WHAMM | −0.1528 | 0.2905 |
| WIPI1 | −0.0072 | 0.3135 |
| XKR8 | −0.0516 | 0.4036 |
| XRCC1 | −0.0982 | 0.3148 |
| YAF2 | −0.0082 | 0.4826 |
| YIF1B | −0.2054 | 0.4641 |
| YWHAB | −0.012 | 0.5026 |
| ZBED1 | −0.0344 | 0.2284 |
| ZC3H12A | −0.0144 | 0.3989 |
| ZC3H3 | −0.0935 | 0.3986 |
| ZCCHC3 | −0.0915 | 0.5589 |
| ZDHHC12 | −0.1493 | 0.415 |
| ZDHHC13 | −0.0375 | 0.2169 |
| ZDHHC16 | −0.0622 | 0.304 |
| ZDHHC6 | −0.215 | 0.4501 |
| ZDHHC9 | −0.0665 | 0.5126 |
| ZFPM1 | −0.2003 | 0.2334 |
| ZFYVE19 | −0.0044 | 0.327 |
| ZFYVE27 | −0.0934 | 0.4456 |
| ZMYND17 | −0.0242 | 0.4521 |
| ZMYND19 | −0.1523 | 0.4117 |
| ZNF296 | −0.0267 | 0.4918 |
| ZNF408 | −0.073 | 0.5245 |
| ZNF444 | −0.0519 | 0.3449 |
| ZNF511 | −0.0713 | 0.3595 |
| ZNF524 | −0.126 | 0.4119 |
| ZNF746 | −0.1494 | 0.2891 |
| ZNF777 | −0.2209 | 0.3061 |
| ZNF784 | −0.0158 | 0.3302 |
| ZNHIT3 | −0.1362 | 0.24 |
| ZP3 | −0.1054 | 0.4221 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting one or more microbial populations or microbial gene expression in a sample, comprising the steps of:
training a model to predict an origin of a nucleotide base-pair sequence;
obtaining reads of transcriptome data of the sample; and
using the model to determine the origin of the reads of the transcriptome data.

2. The method of claim 1, wherein the model is a convolutional neural network with at least one convolutional layer and at least one fully-connected layer.

3. The method of claim 1, wherein the model is trained on a set of human nucleotide base pair sequences, bacterial nucleotide base pair sequences, microbial nucleotide base pair sequences, or a combination thereof.

4. The method of claim 1, wherein the step of training the model comprises the steps of:
obtaining a training set of nucleotide base pair sequences comprising human nucleotide base pair sequences, bacterial nucleotide base pair sequences, microbial nucleotide base pair sequences, or a combination thereof;
labeling the human nucleotide base pair sequences, bacterial nucleotide base pair sequences, or microbial nucleotide base pair sequences in the training set as a human sequence, a bacterial sequence, or a microbial sequence respectively;
training the model to discriminate between human sequence, a bacterial sequence, or a microbial sequence with a first subset of the training set; and
validating the model against a second non-overlapping subset of the training set by comparing a predicted origin of each nucleotide base pair sequences.

5. The method of claim 1, wherein the prediction comprises assigning a score to each nucleotide base pair sequence denoting the relative likelihood of the origin of the nucleotide base pair sequence.

6. The method of claim 1, further comprising the step of assembling the reads determined to be of similar origin into longer sequences.

7. The method of claim 1, wherein the determined origin of reads is selected from one or more of the group consisting of: microbial, bacterial, viral, and human.

8. The method of claim 1, further comprising the step of excluding all reads that map to a human genome.

9. The method of claim 8, wherein the reads are aligned to a database of known microbial sequences.

10. The method of claim 1, wherein the sample is a biological sample from a subject, and the method further comprises comparing the level of the at least one bacteria, at least one bacterial protein, or combination thereof in the biological sample to a comparator, wherein a differential level in the at least one bacteria, at least one bacterial protein, or combination thereof in the biological sample relative to the comparator indicates the subject has, or is at risk for having, cancer.

11. The method of claim 10, wherein the cancer is esophageal cancer.

12. The method of claim 10, wherein the at least one bacteria is one or more bacteria from a genera selected from the group consisting of: Cutibacterium, Sphigomonas, Fictibacillus, Corynebacterium, Bacillus, Gluconacetobacter, Peribacillus, Candidimonas, Burkholderia, Delfita, Halopseodomonas, Methylophilus, and Larkinella.

13. The method of claim 10, wherein:
a decrease in bacteria from the genera selected from the group consisting of Cutibacterium, Sphigomonas, Fictibacillus, and Corynebacterium relative to the comparator indicates the subject has, or is at risk for having, cancer; or
an increase in bacteria from the genera selected from the group consisting of Bacillus, Gluconacetobacter, Peribacillus, Candidimonas, Burkholderia, Delfita, Halopseodomonas, Methylophilus, and Larkinella relative to the comparator indicates the subject has, or is at risk for having, cancer.

14. The method of claim 10, wherein the at least one bacterial protein is one or more selected from the group consisting of: translation elongation factor EF-1 alpha, ferritin, NADHquinone oxidoreductase subunit H, a zincin-like metallopeptidase protein, DNA topoisomerase III, a transposase, a phage replicative protein, acyl-CoA dehydrogenase, LLM-class flavin dependent oxidoreductase, an ABC transporter component, a peptidase, an S49 peptidase, and a phosphatase.

15. The method of claim 14, wherein:
a decrease in bacterial protein selected from the group consisting of translation elongation factor EF-1 alpha, ferritin, NADHquinone oxidoreductase subunit H, a zincin-like metallopeptidase protein, DNA topoisomerase III, and a transposase relative to the comparator indicates the subject has, or is at risk for having, cancer; or
an increase in bacterial protein selected from the group consisting of a phage replicative protein, acyl-CoA dehydrogenase, LLM-class flavin dependent oxidoreductase, an ABC transporter component, a peptidase, an S49 peptidase, and a phosphatase relative to the comparator indicates the subject has, or is at risk for having, cancer.

16. The method of claim 10, further comprising administering to the subject a therapeutic agent to treat or prevent cancer.

17. A method of assessing a prognosis of a subject having cancer comprising:
a. obtaining a biological sample from the subject;
b. measuring the abundance of at least one bacteria, at least one bacterial protein, at least one protein from the subject, or a combination thereof in the biological sample; and
c. comparing the level of the at least one bacteria, at least one bacterial protein, or combination thereof in the biological sample to a comparator, wherein a differential level in the at least one bacteria, at least one bacterial protein, or combination thereof in the biological sample relative to the comparator indicates the prognosis of the subject having cancer.

18. The method of claim 17, wherein the at least one protein from the subject is one or more selected from the group consisting of SAT1, SAT2, FTL, MAP11C3B2, MAP11C3B, and VDAC2, and wherein an increase in the at least one protein from the subject relative to the comparator indicates the subject has a poor prognosis.

19. The method of claim 17, wherein the at least one bacterial protein is one or more selected from the group consisting of: a phage protein, a ribosomal protein, an MFS transporter, a protein linked to mitochondrial function, and an iron-sulfur cluster protein.

20. The method of claim 19, wherein:
a decrease in a protein linked to mitochondrial function, an iron-sulfur protein, or a combination thereof, relative to the comparator indicates the subject has a poor prognosis; or
an increase in at least one bacterial protein selected from the group consisting of a phage protein, a ribosomal protein, an MFS transporter relative to the comparator indicates the subject has a poor prognosis.
